(19) United States
(12) Patent Application Publication
(10) Pub. No.: US 2026/0250261 A1
(43) Pub. Date: Aug. 27, 2026

(54) THIOCTIC ACID BASED LIGANDS

(71) Applicant: The Government of the United States of America, as represented by the Secretary of the Navy, Arlington, VA (US)

(72) Inventors: Kimihiro Susumu, Alexendria, VA (US); Michael Stewart, Springfield, VA (US); Adam Meares, Burke, VA (US); Igor Medintz, Springfield, VA (US); Eunkeu Oh, Alexandria, VA (US)

(73) Assignee: The Government of the United States of America, as represented by the Secretary of the Navy, Arlington, VA (US)

(21) Appl. No.: 19/550,705

(22) Filed: Feb. 26, 2026

Related U.S. Application Data
(60) Provisional application No. 63/763,585, filed on Feb. 26, 2025.

(51) Int. Cl. C07D 339/04; C07C 323/44

(52) U.S. Cl. CPC C07D 339/04; C07C 323/44

(57) ABSTRACT

Disclosed herein are the compounds shown below. R¹ is

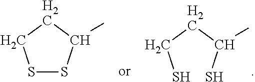

R² is an organic group, R³ is —H, —[(CH₂)₂—O]ₘ—(CH₂)₂—OH or —(CH₂)₂—CO—O—R⁴, at most one of the R³ groups is —H, and R⁴ is an alkyl group or H. The sulfur atoms of these compounds may be bound to nanoparticles, such as semiconducting material, gold, silver, copper, platinum, or palladium.

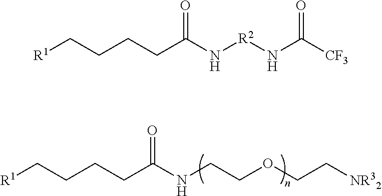

### REFERENCE TO RELATED APPLICATIONS

This application claims the benefit of U.S. Provisional Application No. 63/763,585, filed on Feb. 26, 2025. The provisional application and all other publications and patent documents referred to throughout this nonprovisional application are incorporated herein by reference.

### TECHNICAL FIELD

The present disclosure is generally related to thioctic acid derived compact surface ligands for hydrophilic quantum dots and gold nanoparticles.

### DESCRIPTION OF THE RELATED ART

Thioctic acid (TA) or α-lipoic acid is a known antioxidant that is available as a dietary supplement. It consists of a 1,2-dithiolane ring appended with pentanoic acid. Reduction of thioctic acid forms dihydrolipoic acid (DHLA), which has a dithiol group and has been widely used as a surface binding ligand for preparing colloidally stable semiconductor quantum dots (QDs) and gold nanoparticles (AuNPs). While the terminal carboxyl group of DHLA itself helps improve colloidal stability under basic pH conditions, the protonation of the carboxyl group makes the colloidal form unstable, which significantly limits biological applications. However, this pH limitation can be overcome by modifying the terminal carboxyl group with a variety of functional groups via stable amide bond formation. A wide range of TA and DHLA modified ligands have been designed and their improved utility has been demonstrated; these appended functional groups include poly(ethylene glycol) (PEG) (or oligo(ethylene glycol) (OEG)), amine, biotin, zwitterion, nitrilotriacetic acid (NTA), polymers, and peptides.

There are many benefits for using TA to prepare NP-based ligands. The 1,2-dithiolane ring of TA contains a disulfide unit that intrinsically functions as a protected dithiol. This allows skipping the thiol protection step and simplifies the synthetic strategy and ligand design. In addition, deprotection of the disulfide bond in TA to the dithiol form, DHLA, can be performed under mild reducing conditions, typically using NaBH₄ or tris(2-carboxyethyl)phosphine hydrochloride (TCEP HCl). The DHLA unit also provides an intrinsic improvement in ligand binding strength through a dithiol bidentate chelate effect.

### SUMMARY OF THE INVENTION

Disclosed herein is a compound having the formula below.
R² is an organic group, and R¹ is

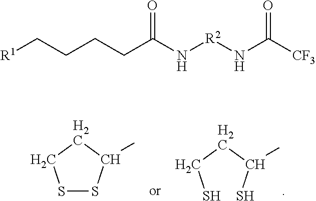

Also disclosed herein is a composition comprising a nanoparticle and the above compound. The sulfur atoms of the compound are bound to the nanoparticle.

Also disclosed herein is a method comprising: providing an amine have the formula

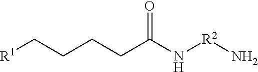

and reacting the amine with ethyl trifluoroacetate to form a compound having the formula below. R¹ and R² are as defined above.

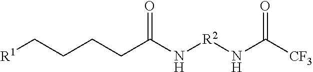

Also disclosed herein is compound having the formula below. R¹ is as defined above. Each R³ is —H, —[(CH₂)₂—O]ₘ—(CH₂)₂—OH or —(CH₂)₂—CO—O—R⁴. At most one of the R³ groups may be —H. R⁴ is an alkyl group or H. The values m and n are positive integers.

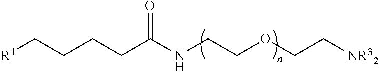

Also disclosed herein is a composition comprising a nanoparticle and the above compound. The sulfur atoms of the compound are bound to the nanoparticle.

### BRIEF DESCRIPTION OF DRAWINGS

A more complete appreciation will be readily obtained by reference to the following Description of the Example Embodiments and the accompanying drawings.

FIGS. 1A-C show chemical structures of the TA- and DHLA-based ligands used herein. The TA based ligands were transformed in situ to the corresponding DHLA derivatives during the ligand exchange procedures.

FIGS. 2A-B show synthetic routes to the series of TA-based ligands.

FIG. 3A shows agarose gel electrophoresis of 520 nm emitting CdSe/CdS/ZnS QDs coated with different ratios of DHLA-EG3-COOH:DHLA-EG3-NH₂: (1) 100% COOH, (2) 90% COOH:10% NH₂, (3) 80% COOH:20% NH₂, (4) 70% COOH:30% NH₂, (5) 60% COOH:40% NH₂, (6) 50% COOH:50% NH₂. Gels were run on 1.0% agarose gel in 1×TBE buffer (pH 8.3) at ~12 V/cm for ~15 min. Gel was imaged under trans UV illumination. Dashed line indicates the location of the wells, and plus and minus signs indicate anode and cathode, respectively.

FIG. 3B shows absorption spectra of the 470 nm emitting ZnSe/Cd_{0.35}Zn_{0.65}S/ZnS QDs purified after the coupling reactions with FITC measured in TRIS buffer (pH 9).

FIG. 3C shows fluorescence spectra of the 470 nm emitting QDs before and after the FITC coupling. The QD samples were measured in TRIS buffer (pH 9) with 400 nm excitation.

FIG. 4A shows agarose gel electrophoresis of 515 nm emitting ZnSe/Cd_{0.4}Zn_{0.6}S/ZnS QDs coated with CL4 derivatives: (1) CL4, (2) DHLA-EG3-CL4, (3) DHLA-PEG600-CL4. Gels were run on 0.8% agarose gel in 1×TBE buffer (pH 8.3) at ~13 V/cm for ~15 min. Gel was imaged under trans UV illumination. Dashed line indicates the location of the wells, and plus and minus signs indicate anode and cathode, respectively.

FIG. 4B shows hydrodynamic size distribution of 515 nm emitting QDs coated with CL4, DHLA-EG3-CL4, and DHLA-PEG600-CL4 in 8 mM NaOH solution.

FIG. 5A shows agarose gel electrophoresis of DHLA-based ligand coated QDs incubated with incremental ratios of BSA. Gels were run on 0.5% agarose gel in 1×TBE buffer (pH 8.3) at ~6.5 V/cm for 30 min. All gels were imaged under trans UV illumination. Dashed line indicates the location of the wells, and plus and minus signs indicate anode and cathode, respectively.

FIG. 5B shows agarose gel electrophoresis of DHLA-based ligand coated AuNPs incubated with incremental ratios of BSA. Gels were run on 0.5% agarose gel in 1×TBE buffer (pH 8.3) at ~6.5 V/cm for 20 min. Dashed line indicates the location of the wells, and plus and minus signs indicate anode and cathode, respectively. While BSA is not visible with white LED backlight (middle and bottom), the faint BSA bands can be traced by trans UV illumination (top). The positions of free BSA bands are indicated by the arrow.

FIG. 6A shows agarose gel electrophoresis of DHLA-based ligand coated 520 nm emitting CdSe/Cd_{0.75}Zn_{0.25}S/ZnS QDs incubated with incremental ratios of BSA. BSA only sample was run together as control for each QD sample. Gels were run on 0.5% agarose gel in 1×TBE buffer (pH 8.3) at ~6.5 V/cm for 30 min. All gels were imaged under trans UV illumination. Dashed line indicates the location of the wells, and plus and minus signs indicate anode and cathode, respectively. Short arrows indicate the positions of free BSA bands.

FIG. 6B shows agarose gel electrophoresis of DHLA-based ligand coated AuNPs incubated with incremental ratios of BSA. BSA only samples were run together as control for each AuNP sample. The trans UV illuminated image for DHLA-TEG (far left) is shown together to locate the position of free BSA, while all the other gels were imaged under white LED exposure. Gels were run on 0.5% agarose gel in 1×TBE buffer (pH 8.3) at ~6.5 V/cm for 30 min. Dashed line indicates the location of the wells, and plus and minus signs indicate anode and cathode, respectively. The arrow indicates the positions of free BSA bands.

FIG. 7A shows PL spectra of AuNCs prepared with terminal hydroxy appended TA-based ligands (TA-EG₂ (lower line in each graph), TA-(OEG)₂ (middle line) and TA-EG3-(OEG)₂ (upper line)) with different [ligand]/[Au] molar ratios: [ligand]/[Au]=(i) 10; (ii) 5; (iii) 2; (iv) 1. The spectra were measured in DI water at 500 nm excitation. For the same Au:ligand ratio, PL intensities were corrected for the absorbances at the excitation wavelength (500 nm).

FIG. 7B shows PL peak wavelength dependent (v) quantum yields and (vi) intensity-weighted average lifetimes of AuNCs prepared with terminal hydroxy appended TA-based ligands with different [ligand]/[Au] molar ratios: TA-EG₂ (filled circle), TA-(OEG)₂ (open square) and TA-EG3-(OEG)₂ (filled triangle).

### DETAILED DESCRIPTION

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

DHLA derivatives and other thiol-based hydrophilic ligands have been utilized to transform as-prepared hydrophobic QDs to hydrophilic QDs via ligand exchange. This QD surface transformation process often requires a large excess of hydrophilic ligands to force nearly complete ligand exchange. Thorough replacement of the original surface ligands is needed to create stable colloidal forms in aqueous media. Therefore, it is highly beneficial to design simple ligand synthetic schemes that have minimal reaction steps and are easy to scale up. Simple yet effective molecular design expands the utility of surface ligands in a large variety of applications.

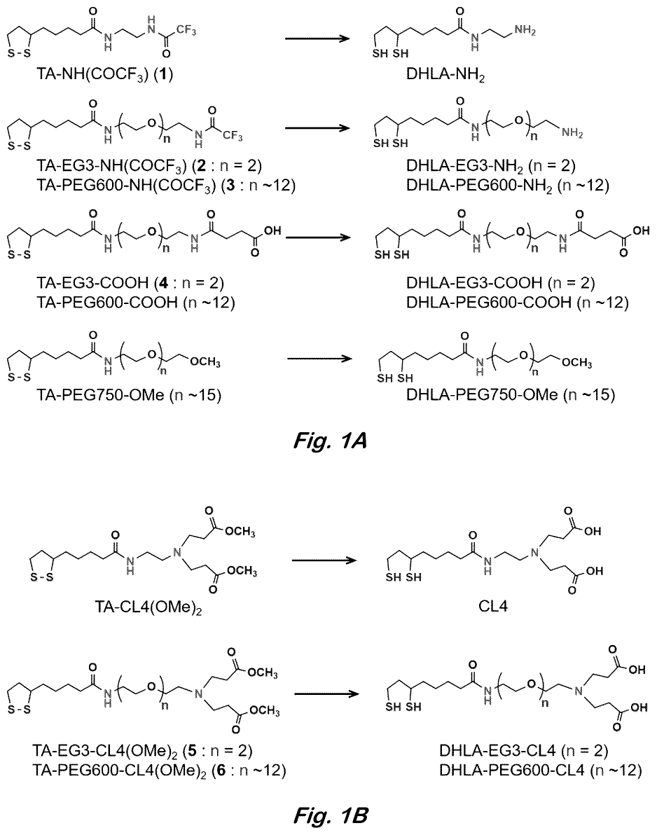

Disclosed herein are efforts to expand and improve the utility of TA and DHLA appended surface ligands for QDs and AuNPs through the modification and refinement of ligand structures (FIGS. 1A-C). This includes: (i) protection of amine-appended TA derivatives to suppress irreversible gelation; (ii) OEG/PEG and zwitterion hybrid ligands to enhance biocompatibility; (iii) hydroxy-terminated OEG/PEG ligands with simple synthetic schemes. The utility of each ligand was demonstrated with QDs and/or AuNPs.

In one synthetic method, the below amine compound

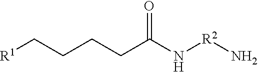

is reacted with ethyl trifluoroacetate to form the below compound.

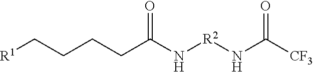

R¹ is a 1,2-dithiolane group or a 1,3-dithiopropyl group and R² is an organic group. The reaction may be performed as described herein or by other methods that may be known in the art that produce the trifluoroacetamide compound. Example compounds include those shown below. The value n is a positive integer, including for example, an integer from 2 to 12.

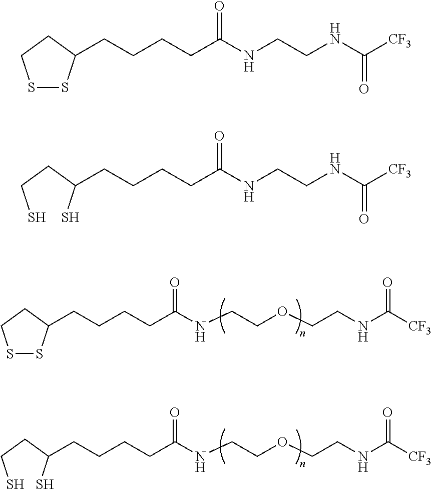

In another embodiment, a compound has the formula below.

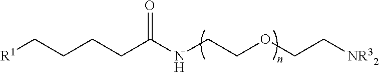

R¹ and n are as defined above. Each R³ is —H, —[(CH₂)₂—O]ₘ, —(CH₂)₂—OH or —(CH₂)₂—CO—O—R⁴, at most one of the R³ groups is —H, R⁴ is an alkyl group or H, and m is a positive integer. Example compounds include those shown below.

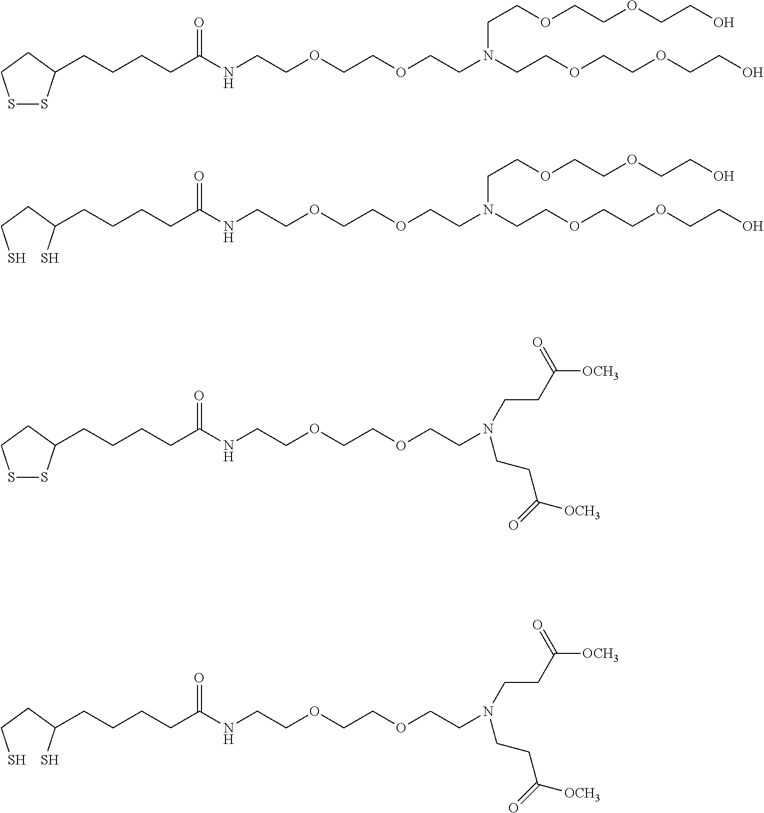 [SMILES: O=C(CCCCC1CCSS1)NCCOCCOCCN(CCOCCOCCO)CCOCCOCCO ; O=C(CCCCC(S)CCS)NCCOCCOCCN(CCOCCOCCO)CCOCCOCCO ; COC(=O)CCN(CCOCCOCCNC(=O)CCCCC1CCSS1)CCC(=O)OC ; COC(=O)CCN(CCOCCOCCNC(=O)CCCCC(S)CCS)CCC(=O)OC]

The sulfur atoms in any of these compounds may be bound to a nanoparticle. In some cases, it may be necessary to first convert the TA group to a DHLA as described herein. Examples of nanoparticle materials include, but are not limited to, a semiconducting material, CdS, ZnS, Ag₂S, PbS, HgS, CdSe, ZnSe, Ag₂Se, PbSe, HgSe, CdTe, ZnTe, Ag₂Te, PbTe, HgTe, CuInS₂, CuInSe₂, InP, InAs, gold, silver, copper, platinum, and palladium. The nanoparticles may comprise alloys of these materials, such as Cd_{0.35}Zn_{0.65}S. The nanoparticles may also have a core and one or more shells of different materials.

The ligands disclosed herein may have a number of potential advantages. First, the trifluoroacetamide protecting group applied to amine-terminated TA-based ligands suppresses irreversible gelation, which was found to be common for the amine-appended TA-based ligands. Both the protection and deprotection steps are straightforward, and the successful in situ deprotection and subsequent ligand exchange on QDs was demonstrated by gel electrophoresis and dye conjugation assays. Second, the TA-based zwitterionic ligands with bis(carboxyethyl)amino group were integrated with oligo(ethylene glycol) (OEG) or poly(ethylene glycol) (PEG) groups to enhance the biocompatibility. Their improved anti-fouling properties were demonstrated by gel electrophoresis assay with bovine serum albumin (BSA). Also, hydroxy-terminated branched TA-based ligands were synthesized in simple minimal steps as alternatives of hydroxy-terminated TA-based OEG/PEG ligands, which require tedious purification steps. The utility of the compact hydroxy-terminated ligands was further demonstrated by (i) the gel electrophoresis assay to explore the anti-fouling properties and (ii) photoluminescence tuning in the direct aqueous synthesis of luminescent gold nanoclusters (AuNCs). Inherent benefits of the ligand design were demonstrated beyond QDs as AuNPs functionalized with the same compact ligand series showed similar colloidal properties.

The synthetic schemes and technical procedures of each ligand were well optimized. Each ligand can be synthesized in minimal synthetic steps and simple column purification procedures. A trifluoroacetamide protecting group significantly suppresses gelation of amine-appended TA ligands and extends the shelf life. Some of the amine-appended TA based ligands are commercially available. However, their actual processability and utility are highly questionable because amine-appended TA based ligands tend to form gels in an irreversible manner. Deprotection of trifluoroacetamide protecting group can be done in mild conditions. This allows in situ deprotection during ligand exchange with NPs, and the amine-appended TA-based ligands can be quickly ready for subsequent synthetic steps.

TA-OEG/PEG-CL4 ligands can be synthesized in a manner similar to the original CL4 ligand. The combination of known hydrophilicity and biocompatibility of both OEG/PEG and zwitterionic CL4 functional groups helps further improve the colloidal stability and anti-fouling properties compared to CL4 ligand itself.

TA-(OEG)₂ and TA-EG3-(OEG)₂ ligands can be synthesized in a few synthetic steps without protecting reactive hydroxy terminal groups. The entire synthetic procedures are simpler and less laborious than those of TA-OEG/PEG series that have been commercially available.

The TA based ligands can be utilized for direct aqueous synthesis of near IR emitting AuNCs. The PL peak positions can be simply tuned by (i) [ligand]/[Au] molar ratios and (ii) the size of TA derived ligands.

TA-based surface ligands were synthesized for use with both QDs and AuNPs to further extend their utility and/or improve the biocompatibility of the ligands previously developed, and their versatile use was successfully demonstrated. Importantly, the material cost and simple synthetic procedures were taken into account to design these new ligands. Gel formation has been a universal issue for amine-terminated TA-based ligands and made the post-purification material handling difficult. The protection of the ligand amine with a trifluoroacetamide group mitigates this using a mild and simple condition, and deprotection can be easily done in situ during the NP ligand exchange step due to its intrinsically labile character. The integration of both PEG (or OEG) and bis(carboxyethyl)amine groups (CL4 functional group) in TA-based ligands was simply designed by the use of diamino PEG (or OEG) derivatives in place of ethylenediamine without creating any extra steps. The hybridization of PEG (or OEG) and zwitterionic units helps enhance the biocompatibility of ligand-coated NPs. The hydroxy-terminal TA-based ligands were designed with minimal synthetic steps and simple procedures. The anti-fouling properties and the utility to control the core growth of AuNCs are certainly beneficial to further explore this ligand series since the synthesis of most other hydroxy-terminated ligands is often laborious, ends up with lower yields, and is not cost efficient. Given the exceptionally facile synthetic schemes along with the excellent aqueous colloidal stability and anti-fouling properties imparted to QDs and AuNPs, the ligand series developed in the present study will be able to further expand the utility of QDs and AuNPs in many biological applications.

Potential advantages include:
1) Each ligand can be synthesized in minimal synthetic steps and simple column purification procedures, which would be highly beneficial for commercialization aspect.
2) A trifluoroacetamide protecting group significantly suppresses gelation of amine-appended TA ligands and extends the shelf life.
3) Deprotection of trifluoroacetamide protecting group can be done in mild conditions, and this allows in situ deprotection during ligand exchange with NPs.
4) TA-OEG/PEG-CL4 ligands can be synthesized in a manner similar to the original CL4 ligand and help improve the colloidal stability and anti-fouling properties.
5) TA-(OEG)₂ and TA-EG3-(OEG)₂ ligands can be synthesized in a few synthetic steps without protecting reactive hydroxy terminal groups. The entire synthetic procedures are simpler and less laborious than those of TA-OEG/PEG series that have been commercially available.
6) The TA based ligands can be utilized for direct aqueous synthesis of near IR emitting AuNCs. The PL peak positions can be tuned by [ligand]/[Au] molar ratios and the size of TA derived ligands.

The following examples are given to illustrate specific applications. These specific examples are not intended to limit the scope of the disclosure in this application.

Quantum dots—470 nm emitting ZnSe/Cd_{0.35}Zn_{0.65}S/ZnS core-shell QDs and 515 nm emitting ZnSe/Cd_{0.4}Zn_{0.6}S/ZnS core-shell QDs were synthesized as previously described (Susumu et al., Chem. Mater. 2017, 29, 7330-7344). ~520 nm emitting CdSe/CdS/ZnS core-shell QDs were synthesized as previously described (Green et al., ACS Nano 2022, 16, 20693-20704, Supporting Information). Typical synthetic procedures for ~520 nm emitting CdSe—CdZnS—ZnS core-shell QDs are described below.

CdSe core synthesis—Cadmium acetylacetonate (0.311 g, 1.0 mmol) was mixed with 5.0 mL of tri-n-octylphosphine (TOP) in a 20-mL vial, which was sealed with a septum. The mixture was degassed under vacuum at ~100° C. for 10 min, backfilled with N₂ and stirred at ~100° C. until the Cd precursor was dissolved. Tri-n-octylphosphine oxide (TOPO, 90%, 1.0 g), TOP (13 mL), hexadecylamine (HDA, 5.0 g), tetradecylphosphonic acid (TDPA, 0.278 g, 1.0 mmol), and Se (0.363 g, 4.60 mmol) were loaded into a 50-mL three-neck round-bottom flask. The reaction mixture was degassed under vacuum at ~100° C. for 30 min, backfilled with N₂, and subsequently heated to 250° C. The heating mantle was removed, then the Cd precursor solution was swiftly injected into the reaction mixture under vigorous stirring at 250° C. When the reaction mixture reached ~60° C., n-butanol was added to prevent solidification of the reaction mixture. The reaction mixture was aliquoted to 40-mL vials. Excess acetone was added to each vial to flocculate the QDs. The mixtures were centrifuged at 4,300 rpm for 5 min. The supernatant was discarded, and the QD pellets were dissolved in a minimum amount of CHCl₃. This cleaning procedure was repeated one more time. The final CdSe QD concentration was estimated following literature methods (Jasieniak et al., J. Phys. Chem. C 2009, 113, 19468-19474).

Overcoating of CdSe core with CdZnS ZnS shells—TOPO (90%, 15.0 g), oleylamine (5.0 mL), tri-n-butylphosphine (TBP, 8.0 mL) and the CdSe QD core (9.0×10⁻⁷ mol in CHCl₃ solution) were loaded into a 100-mL three-neck round-bottom flask. The reaction mixture was degassed under vacuum at 100° C. to remove CHCl₃ and other volatiles and backfilled with N₂. The amount of shell precursors used for the overcoating was calculated following literature procedures (Chen et al., Chem. Mater. 2010, 22, 1437-1444). For the coating of a half monolayer of CdZnS, a mixture of CdMe₂, ZnEt₂ and hexamethyldisilathiane ((TMS)₂S) in TBP was added dropwise at 140° C. Subsequently a mixture of ZnEt₂ and (TMS)₂S in TBP was added dropwise starting at 150° C., and the reaction temperature gradually raised to 180° C. during the ZnS precursor addition. A 0.1 M Zn oleate solution (prepared from ZnO and oleic acid in 1:8 ratio in ODE) was further added to create a zinc rich surface, and the reaction mixture was annealed at 230° C. for 1 h.

Gold nanoparticle synthesis—The AuNPs were synthesized by a seeded growth method using 3.2 nm seed AuNPs, with some modifications of the method previously reported (Breger et al., Bioconjugate Chem. 2019, 30, 2060-2074; Jana et al., Langmuir 2001, 17, 6782-6786). First, 3.2 nm seed AuNPs were synthesized with sodium citrate as a ligand and NaBH₄ as a reducing agent. 125 μL (1.25×10⁻⁵ mol) of a 100 mM tetrachloroauric (III) acid (HAuCl₄·3H₂O) aqueous stock solution and 125 μL (2.5×10⁻⁵ mol) of 200 mM sodium citrate stock solution were dissolved in 50 mL of deionized (DI) water, and the mixture was stirred at room temperature for 5 min. 125 μL (1.25×10⁻⁴ mol) of 1 M NaBH₄ stock solution in DI water was then added to the mixture with vigorous stirring. The reaction mixture was kept stirred vigorously for 30 min.

To grow the seed AuNPs larger, the growth solution was prepared with 100 μL (1.0×10⁻⁵ mol) of a 100 mM tetrachloroauric (III) acid (HAuCl₄·3H₂O) aqueous stock solution and 100 μL (2.0×10⁻⁵ mol) of a 200 mM sodium citrate stock solution that were dissolved in 50 mL of DI water. The freshly prepared growth solution (25 mL) was then mixed with 30 mL of 3.2 nm seed AuNPs with vigorous stirring. After 2 min, 100 μL (2.0×10⁻⁵ mol) of 200 mM L-ascorbic acid stock solution in DI water was added to the mixture with vigorous stirring. The reaction mixture was stirred at room temperature for 30 min and kept without stirring for an additional 24 h for the complete reaction. Reaction completion was confirmed by the red shift of the AuNP surface plasmon band peak and the corresponding decrease of the ascorbic acid and aurate peaks in the near UV region (<300 nm) using UV-vis absorption spectroscopy. The final average size (7.8±1.5 nm) was confirmed by TEM.

Ligand synthesis—Among a series of NP surface ligands in the present study, TA-PEG750-OMe, TA-PEG600-NH₂, TA-PEG600-COOH, TA-CL4(OMe)₂ and TA-EG₂ were synthesized as previously described (Mei et al., J Mater. Chem. 2008, 18, 4949-4958; Mei et al., Nat. Protoc. 2009, 4, 412-423; Susumu et al., J. Am. Chem. Soc. 2007, 129, 13987-13996; Susumu et al., Nat. Protoc. 2009, 4, 424-436; Susumu et al., J. Am. Chem. Soc. 2011, 133, 9480-9496). The synthetic procedures of Compounds 4, 6, 11-18 can be found elsewhere (Susumu et al., Chem. Mater. 2025, 37, 4375-4392, Supporting Information; Brown et al., RSC Adv., 2015, 5, 93089-93094).

N-[2-[(2′,2′,2′-trifluoroacetyl)amino]ethyl]-1,2-dithiolane-3-pentanamide 1—Thioctic acid (2.00 g, 9.69×10⁺³ mol) and 1,1′-carbonyldiimidazole (CDI, 1.88 g, 1.16×10⁻² mol) were added to a 100-mL round-bottom flask, and the mixture was purged with N₂. 30 mL of CHCl₃ was added to the mixture by syringe, and the reaction mixture was stirred at room temperature for 1 h under N₂. The reaction mixture was then transferred to an addition funnel and 15 mL of CHCl₃ was mixed in. The solution was added dropwise over 1 h to a mixture of ethylenediamine (6.4 mL, 9.57×10⁻² mol) and 30 mL of CHCl₃ in a 250-mL two-neck round-bottom flask at room temperature under N₂. The reaction mixture was stirred at room temperature for 3.5 h in total and transferred to a separatory funnel. DI water (~100 mL) was added to the mixture, and the CHCl₃ layer was collected. The aqueous layer was further washed with CHCl₃ (3 times). The combined organic layers were washed with 0.5M NaHCO₃ solution (100 mL) and dried over Na₂SO₄. The inorganic salt was filtered off and the filtrate was concentrated to ~20 mL. The crude product solution was chromatographed on silica gel with CHCl₃:MeOH (4:1) as the eluent. The product fractions were concentrated to ~30 mL and MeOH (30 mL) was mixed in. Ethyl trifluoroacetate (1.20 mL, 1.01×10⁻² mol) in MeOH (5.0 mL) was added dropwise at room temperature under N₂, and the reaction mixture was stirred for 2 h. The solvent and excess reagent were evaporated to obtain the product as yellow solid. Yield=2.073 g (62% based on 2.00 g of thioctic acid). ¹H NMR (400 MHz, CDCl₃): δ 7.89 (br s, 1H, NH), 5.91 (br s, 1H, NH), 3.52-3.62 (m, 1H), 3.44-3.52 (m, 4H, —CH₂—), 3.07-3.23 (m, 2H), 2.41-2.52 (m, 1H), 2.24 (t, 2H, J=9.9 Hz, —CH₂—CO—), 1.85-1.97 (m, 1H), 1.6-1.8 (m, 4H), 1.4-1.53 (m, 2H). ¹⁹F NMR (376 MHz, CDCl₃): δ −76.79 (s). Product was confirmed by electrospray ionization mass spectrometry (ESI MS) with a mass-to-charge ratio (m/z) of 382.97 (M+K)⁺/theoretical monoisotopic mass (M+K)⁺ was 383.048.

N-[2-[2-[2-[(2′,2′,2′-trifluoroacetyl)amino]ethoxy]ethoxy]ethyl]-1,2-dithiolane-3-pentanamide 2—Thioctic acid (2.00 g, 9.69×10⁻³ mol) and CDI (2.05 g, 1.26×10⁻² mol) were added to a 100-mL round-bottom flask, and the mixture was purged with N₂. 20 mL of CHCl₃ was added to the mixture by syringe, and the reaction mixture was stirred at room temperature for 1 h under N₂. The reaction mixture was then transferred to an addition funnel and 10 mL of CHCl₃ was mixed in. The solution was added dropwise over 45 min to a mixture of 1,2-bis(2-aminoethoxy)ethane (14.50 g, 9.78×10⁻² mol) and 30 mL of CHCl₃ in a 250-mL two-neck round-bottom flask at room temperature under N₂. The reaction mixture was stirred at room temperature for 16.5 h and transferred to a separatory funnel. 1M NaHCO₃ solution (~100 mL) was added to the mixture, and the CHCl₃ layer was collected. The aqueous layer was further washed with CHCl₃ (2 times). The combined organic layers were washed with 1M NaHCO₃ solution (100 mL) and dried over Na₂SO₄. The inorganic salt was filtered off and the filtrate was concentrated to ~20 mL. The crude product solution was chromatographed on silica gel with CHCl₃:MeOH (5:1→4:1) as the eluent. The product fractions were concentrated to ~20 mL, and MeOH (30 mL) was mixed in. Ethyl trifluoroacetate (1.40 mL, 1.18×10⁻² mol) in MeOH (7.0 mL) was added dropwise at room temperature under N₂, and the reaction mixture was stirred for 20 h. The solvent and excess reagent were evaporated to obtain the product as pale yellow oil. Yield=3.448 g (82% based on 2.00 g of thioctic acid). ¹H NMR (400 MHz, CDCl₃): δ 7.07 (br s, 1H, NH), 5.86 (br s, 1H, NH), 3.52-3.7 (m, 11H), 3.43-3.50 (m, 2H, —CH₂—), 3.07-3.23 (m, 2H), 2.42-2.51 (m, 1H), 2.20 (t, 2H, J=7.4 Hz, —CH₂—CO—), 1.86-1.96 (m, 1H), 1.6-1.8 (m, 4H), 1.4-1.54 (m, 2H). ¹⁹F NMR (376 MHz, CDCl₃): δ −76.62 (s). ESI MS: m/z 470.98 ([M+K]⁺) (calculated 471.100).

TA-PEG600-trifluoroacetamide 3—TA-PEG600-NH₂ (Susumu et al., J. Am. Chem. Soc. 2007, 129, 13987-13996; Susumu et al., Nat. Protoc. 2009, 4, 424-436) (2.828 g, 3.64×10⁻³ mol) was mixed with MeOH (10 mL) and Et₃N (0.75 mL, 5.4×10⁻³ mol) and the mixture was purged with N₂. Ethyl trifluoroacetate (1.30 mL, 1.09×10⁻² mol) in MeOH (10 mL) was added dropwise at room temperature under N₂. The reaction mixture was stirred at room temperature overnight under N₂. The solvent and excess reagent were evaporated. The residue was dissolved in ethyl acetate, washed twice with brine and dried over Na₂SO₄. The inorganic salt was filtered off and the solvent was evaporated to obtain the product as pale yellow oil. Yield=2.766 g (87% based on 2.828 g of TA-PEG600-NH₂). ¹H NMR (400 MHz, CDCl₃): δ 7.81 (br s, 1H, NH), 6.31 (br s, 1H, NH), 3.5-3.7 (m), 3.41-3.48 (m, 2H, —CH₂—), 3.08-3.21 (m, 2H), 2.41-2.51 (m, 1H), 2.19 (t, 2H, J=7.2 Hz, —CH₂—CO—), 1.86-1.96 (m, 1H), 1.6-1.8 (m, 4H), 1.4-1.52 (m, 2H). ¹⁹F NMR (376 MHz, CDCl₃): δ −76.49 (s). ESI MS: m/z 867.11 (n=11), 911.13 (n=12), 955.15 (n=13) ([M+K]⁺) (calculated 867.336 (n=11), 911.362 (n=12), 955.389 (n=13), where n refers to the number of ethylene oxide units within PEG chain as defined in the chemical structure in FIG. 1A).

TA-EG3-CL4(OMe)₂ 5—Thioctic acid (2.119 g, 1.03×10⁻² mol) and CDI (2.31 g, 1.42×10⁻² mol) were added to a 100-mL round-bottom flask, and the mixture was purged with N₂. 20 mL of CHCl₃ was added to the mixture by syringe, and the reaction mixture stirred at room temperature for 1 h under N₂. The reaction mixture was then transferred to an addition funnel and 5 mL of CHCl₃ was mixed in. The solution was added dropwise over 30 min to a mixture of 1,2-bis(2-aminoethoxy)ethane (12.00 g, 8.10×10⁻² mol) and 20 mL of CHCl₃ in a 250-mL two-neck round-bottom flask at room temperature under N₂. The reaction mixture was stirred at room temperature for 17 h and transferred to a separatory funnel. 1M NaHCO₃ solution (~100 mL) was added to the mixture, and the CHCl₃ layer was collected. The aqueous layer was further washed twice with CHCl₃. The combined organic layers were washed with 1M NaHCO₃ solution (100 mL) and dried over Na₂SO₄. The inorganic salt was filtered off. The filtrate was concentrated to ~20 mL, and MeOH (20 mL) was added to the filtrate solution. To the combined filtrate was added methyl acrylate (4.00 mL, 4.44×10⁻² mol) in MeOH (25 mL) dropwise. The reaction mixture was stirred at room temperature for 3 days under N₂. The solvent and excess methyl acrylate were evaporated. The residue was chromatographed on silica gel with CHCl₃:MeOH (15:1). Yield=4.438 g (85% based on 2.119 g of thioctic acid). ¹H NMR (400 MHz, CDCl₃): δ 6.15 (br s, 1H, NH), 3.67 (s, 6H, —OCH₃), 3.51-3.63 (m, 9H), 3.42-3.50 (m, 2H, —OCH₂—), 3.07-3.22 (m, 2H), 2.84 (t, 4H, J=7.2 Hz, —NCH₂—), 2.68 (t, 2H, J=6.2 Hz, —NCH₂—), 2.42-2.51 (m, 1H), 2.47 (t, 4H, J=7.0 Hz, —CH₂—CO—), 2.20 (t, 2H, J=7.4 Hz, —CH₂—CO—), 1.86-1.96 (m, 1H), 1.64-1.76 (m, 4H), 1.4-1.52 (m, 2H). ESI MS: m/z 509.10 ([M+H]⁺) (calculated 509.235).

N,N-Bis{2-[2-(2-hydroxyethoxy)ethoxy]ethyl}-N′-(tert-butoxycarbonyl)ethylenediamine 7—A mixture of 2-[2-(2-chloroethoxy)ethoxy]ethanol (20.4 g, 0.121 mol), NaI (30.0 g, 0.200 mol), Na₂CO₃ (90.0 g, 0.849 mol) and dry CH₃CN (500 mL) was refluxed for 7.5 h under N₂, then N-(tert-butoxycarbonyl)ethylenediamine (8.30 mL, 5.24×10² mol) was added. The reaction mixture was further refluxed for 64 h under N₂. After cooling, the white solid was filtered off and washed with CH₃CN. The filtrate was evaporated and DI water (~150 mL) was added to the residue. The aqueous layer was washed twice with ether, and the product was extracted with CHCl₃ (4 times). The combined CHCl₃ layers were dried over Na₂SO₄. The inorganic salt was filtered off and the solvent was evaporated to yield the product as oil. Yield=20.67 g (93% based on 8.30 mL of N-(tert-butoxycarbonyl)ethylenediamine). ¹H NMR (400 MHz, CDCl₃): δ 5.71 (br s, 1H, NH), 3.73 (t, 4H, J=5.8 Hz, OCH₂), 3.58-3.70 (m, 12H, OCH₂), 3.55 (t, 4H, J=7.4 Hz, OCH₂), 3.17 (m, 2H, NHCH₂), 2.73 (t, 4H, J=7.2 Hz, NCH₂), 2.62 (t, 2H, J=7.6 Hz, NCH₂), 1.45 (s, 9H, t-Bu). ESI MS: m/z 425.20 ([M+H]⁺) (calculated 425.286).

N,N-Bis{2-[2-(2-hydroxyethoxy)ethoxy]ethyl}ethylenediamine 8—A mixture of 7 (8.145 g, 1.92×10⁻² mol) and 4M HCl in dioxane (25 mL) was stirred at room temperature for 1 h under N₂. The solvent was evaporated. DI water (80 mL) was added to the residue. The aqueous layer was washed twice with ether, then basified with NaOH (pH>13), and saturated with NaCl. The product was extracted several times with CHCl₃. The combined organic layers were dried over Na₂SO₄. The inorganic salt was filtered off and the filtrate was evaporated to obtain the product as oil. Yield=5.817 g (93% based on 8.145 g of 7). ¹H NMR (400 MHz, CDCl₃): δ 3.75 (t, 4H, J=5.8 Hz, OCH₂), 3.64 (m, 8H, OCH₂), 3.60 (t, 4H, J=5.8 Hz, OCH₂), 3.52 (t, 4H, J=6.8 Hz, OCH₂), 2.84 (t, 2H, J=7.4 Hz, NCH₂), 2.72 (t, 4H, J=6.8 Hz, NCH₂), 2.66 (t, 2H, J=7.4 Hz, NCH₂). ESI MS: m/z 325.16 ([M+H]⁺) (calculated 325.234).

TA-(OEG)₂ 9—A mixture of thioctic acid (0.615 g, 2.98×10⁻³ mol) and EEDQ (1.10 g, 4.45×10⁻³ mol) in CH₂Cl₂ (6.0 mL) was stirred at room temperature for 15 min under N₂. To the reaction mixture was added 8 (1.017 g, 3.13×10⁻³ mol) in CH₂Cl₂ (7.0 mL). The reaction mixture was stirred at room temperature for 15 h under N₂. The solvent was evaporated, and the residue was chromatographed on silica gel with CHCl₃:MeOH (10:1) as an eluent to purify the product. Yield=1.228 g (80% based on 0.615 g of thioctic acid). ¹H NMR (400 MHz, CDCl₃): δ 7.22 (br s, 1H, NH), 3.72 (t, 4H, J=5.8 Hz, OCH₂), 3.5-3.7 (m, 17H, OCH₂ and CH), 3.32 (q, 2H, J=7.2 Hz, NHCH₂), 3.06-3.23 (m, 2H), 2.71 (t, 4H, J=6.9 Hz, NCH₂), 2.61 (t, 2H, J=7.2 Hz, NCH₂), 2.40-2.52 (m, 1H), 2.22 (t, 2H, J=9.9 Hz, CH₂CO), 1.84-1.97 (m, 1H), 1.58-1.78 (m, 4H), 1.37-1.55 (m, 2H). ESI MS: m/z 513.15 ([M+H]⁺) (calculated 513.267).

TA-EG3-(OEG)₂ 10—A mixture of 2-[2-(2-chloroethoxy)ethoxy]ethanol (3.618 g, 2.15×10⁻² mol), NaI (5.32 g, 3.55×10⁻² mol), Na₂CO₃ (7.10 g, 6.70×10⁻² mol) and dry CH₃CN (50 mL) was refluxed for 5.5 h under N₂, then cooled and set aside. Complete conversion to 2-[2-(2-iodoethoxy)ethoxy]ethanol was determined by TLC.

Thioctic acid (2.00 g, 9.69×10⁻³ mol) and CDI (2.13 g, 1.31×10⁻² mol) were added to a 100-mL round-bottom flask, and the mixture was purged with N₂. 20 mL of CHCl₃ was added to the mixture by syringe, and the reaction mixture was stirred at room temperature for 2 h under N₂. The reaction mixture was then transferred to an addition funnel, and 5 mL of CHCl₃ was mixed in. The solution was added dropwise over 1 h to a mixture of 1,2-bis(aminoethoxy)ethane (11.573 g, 7.81×10⁻² mol) and 20 mL of CHCl₃. The reaction mixture was stirred at room temperature for 4.5 h under N₂. 1M NaHCO₃ aqueous solution (~100 mL) was added to the reaction mixture, and the CHCl₃ layer was collected. The aqueous layer was further washed twice with CHCl₃. The combined organic layers were washed with 1M NaHCO₃ solution (100 mL) and dried over Na₂SO₄. The inorganic salt was filtered off and the filtrate was concentrated to ~10 mL. The crude product solution was chromatographed on silica gel with CHCl₃:MeOH (4:1) as the eluent. The solvent of the product fractions was evaporated. The residue was mixed with dry CH₃CN (10 mL) and added to the solution of 2-[2-(2-iodoethoxy)ethoxy]ethanol prepared above.

The reaction mixture was refluxed for 46 h under N₂. After cooling, the white solid was filtered off, and the solvent was evaporated. The residual oil was mixed with 100 mL of DI water and washed twice with ethyl acetate. Then the product was extracted with CHCl₃ (4 times). The combined organic layers were dried over Na₂SO₄. The white solid was filtered off, and the solvent was evaporated. The residue was chromatographed on silica gel with CHCl₃:MeOH (10:1). Yield=2.400 g (41% based on 2.00 g of thioctic acid). ¹H NMR (400 MHz, CDCl₃): δ 6.60 (br s, 1H, NH), 3.72 (t, 4H, J=4.4 Hz, OCH₂), 3.52-3.68 (m, 25H, OCH₂ and CH), 3.41-3.48 (q, 2H, J=5.0 Hz, NHCH₂), 3.07-3.22 (m, 2H), 2.79 (m, 6H, NCH₂), 2.41-2.51 (m, 1H), 2.20 (t, 2H, J=7.4 Hz, CH₂CO), 1.85-1.96 (m, 1H), 1.59-1.76 (m, 4H), 1.39-1.55 (m, 2H). ESI MS: m/z 601.19 ([M+H]⁺) (calculated 601.319).

Ligand exchange onto quantum dots—(i) Ligand exchange with TA-PEG750-OMe:TA-PEG600-NHCOCF₃ (95:5)—Typical procedures for ligand exchange are as follows. QDs coated with native hydrophobic ligands (~4.0 nmol in stock solution) were transferred from the stock solution to a 20-mL vial, and acetonitrile and isopropanol were added to flocculate the QDs. The QD solution was centrifuged (3,800 rpm, 5 min) and the supernatant was discarded. 0.1M ZnCl₂ in EtOH (0.3 mL), 2-(2-aminoethoxyl)ethanol (0.4 mL) and CHCl₃ (0.3 mL) were mixed with the QD pellet. The vial was sealed with a septum poked with a syringe needle. The QD mixture was stirred at 105° C. for 2 h under N₂ flow. After cooling, excess ethyl acetate was added to flocculate the QDs. The QD mixture was centrifuged (3,500 rpm, 8 min), and the supernatant was discarded. The QD pellet was mixed with MeOH (0.3 mL), and the solution was sealed with a septum under N₂. In a separate vial, a mixture of TA-PEG750-OMe (63.6 mg, 6.88×10⁻⁵ mol) and TA-PEG600-NHCOCF₃ (3.2 mg, 3.67×10⁻⁶ mol) were dissolved in EtOH (0.4 mL), and NaBH₄ (14.2 mg, 3.75×10¹ mol) was added. The reaction mixture was stirred at room temperature for 1 h under N₂. 4M HCl was then added dropwise to the reaction mixture to adjust the pH to 7~8. Then the ligand solution was injected to the QD solution prepared above. The reaction mixture was stirred at 40° C. for 2 h and at room temperature for 14 h under N₂. MeOH was evaporated, and ethyl acetate and hexanes were added to flocculate the QDs. The reaction mixture was centrifuged (3,500 rpm, 8 min). The supernatant was discarded and the QD pellet was dissolved in DI water. The QD solution was then filtered through a Millex-LCR membrane filter (pore size 0.45 μm, Millipore) and transferred to a centrifugal spin dialyzer (Amicon Ultra 50K, Millipore). The mixture was diluted with DI water and centrifuged at 3,800 rpm for 5~10 min, and the clear filtrate was discarded. To remove excess unbound ligands and other byproducts, the QD dispersion was subject to a few additional rounds of centrifugation with DI water. The concentrated QD solution was finally filtered through a Millex-LG membrane filter (pore size 0.20 μm, Millipore).

(ii) Ligand exchange with CL4 series—Ligand exchange with CL4 series was performed as previously described with some modification (Susumu et al., J. Am. Chem. Soc. 2011, 133, 9480-9496; Susumu et al., Chem. Mater. 2017, 29, 7330-7344).

(iii) Ligand exchange with hydroxy-terminated ligands—Typical procedures for ligand exchange are as follows. QDs coated with native hydrophobic ligands (~5.0 nmol in stock solution) were flocculated by mixing with isopropanol and acetonitrile. The mixture was centrifuged at 3,800 rpm for 5 min. The clear supernatant was discarded. The QD pellet was mixed with 2-(2-aminoethoxyl)ethanol (0.30 mL), CHCl₃ (0.30 mL) and methanol (0.40 mL). The reaction mixture was sealed with a septum and stirred at 50° C. for 1 h under N₂. After cooling, excess ethyl acetate was added to the mixture to flocculate the QDs. The mixture was centrifuged at 3,800 rpm for 5 min, and the supernatant was discarded. The QD pellet was dissolved in 0.2 mL of MeOH, and the solution was sealed with a septum under N₂. In a separate vial, TA-(OEG)₂ (52.3 mg, 1.02×10⁻⁴ mol) was dissolved in MeOH (0.3 mL), and NaBH₄ (16.3 mg, 4.3×10⁻⁴ mol) was added. The reaction mixture was stirred at room temperature for 1 h under N₂. 4M HCl was then added dropwise to the reaction mixture to adjust the pH to ~8. 0.1M Zn(NO₃)₂ in EtOH (0.50 mL, 5×10⁻⁵ mol) was mixed in a vial, and the mixture was stirred at 44° C. for 1 h 45 min under N₂. The ligand solution was then injected by a syringe into the QD solution prepared above. The reaction mixture was stirred at 40° C. for 2.5 h under N₂. After cooling, the solvent was evaporated, and ethyl acetate was added to the QD pellet. The mixture was centrifuged at 3,800 rpm for 5 min, and the supernatant was discarded. The QD pellet was dissolved in DI water. The QD solution was filtered through a Millex-LCR membrane filter (pore size 0.45 μm, Millipore) and transferred to a centrifugal spin dialyzer (Amicon Ultra 50K, Millipore). The mixture was diluted with DI water and centrifuged at 3,800 rpm for 5~10 min, and the clear filtrate was discarded. To remove excess unbound ligands and other byproducts, the QD dispersion was subject to a few additional rounds of centrifugation with DI water, followed by filtration through a Millex-LG membrane filter (pore size 0.20 μm, Millipore).

Ligand exchange onto gold nanoparticles—For ligand exchange with AuNPs, the ligands were used without the ring opening step of the dithiolane groups due to the much stronger intrinsic Au—S interaction. Ligand exchange of the CL4 series with AuNPs were performed as described before. Typical procedures for the ligand exchange of hydroxy-terminated ligands are as follows. The ligand solution (in case of ligand TA-(OEG)₂, the ligand was dissolved in 1.0 mL of 50 mM NaOH) was added to citrate-stabilized AuNPs (~7.8 nm diameter, 5.0 mL, 5×10⁻¹⁰ mol), and the reaction mixture was stirred at room temperature overnight. The aqueous solution was filtered through a Millex-LCR membrane filter (pore size 0.45 μm, Millipore) and transferred to a centrifugal spin dialyzer (Amicon Ultra 30K, Millipore). The mixture was diluted with DI water and centrifuged at 3,800 rpm for 5-10 min and the clear filtrate was discarded. To remove excess unbound ligands and other byproducts, the AuNP dispersion was subject to a few additional rounds of centrifugation with DI water.

Gold nanocluster synthesis—AuNCs were synthesized following previously published procedures with some modifications (Oh et al., Part. Part. Syst. Charact. 2013, 30, 453-466). Briefly, 50 μL (1.0×10⁻⁴ mol) of 2M NaOH and 100~1000 μL (1.0~10×10⁻⁵ mol) of 100 mM ligand aqueous stock solution and 100 μL (1.0×10⁻⁵ mol) of 100 mM tetrachloroauric (III) acid (HAuCl₄·3H₂O) aqueous solution were dissolved in 25 mL of deionized water, and the mixture was stirred at room temperature for 5 min. 150 μL (3.0×10⁻⁵ mol) of 200 mM NaBH₄ solution in deionized water was then added dropwise with vigorous stirring. The stirring rate was lowered after 30 min, and the mixture was left stirring in the dark for 20~24 h. The dispersion was then purified from free ligands by two or three cycles of centrifugation using a centrifugal membrane filtration device (10 K molecular weight cut-off, Millipore Corporation, Billerica, MA). AuNC concentration was determined as previously reported (Oh et al., Sci. Rep. 2016, 6, 35538).

Gel electrophoresis for anti-fouling study—QD and AuNP samples were prepared in 1×PBS solution. For the anti-fouling test with BSA, QDs (5 μmol) or AuNPs (5 μmol) were mixed with increasing ratios of BSA in 1×PBS at molar ratios ranging from 0 to 1,500 and were allowed to incubate at room temperature for 30 min. Each sample was mixed with 25% glycerol loading buffer, loaded in a well, and then run on 0.5% agarose gel using 1×tris borate EDTA buffer (TBE, 90 mM Tris, 90 mM boric acid, 2 mM EDTA, pH 8.3). The experiments were conducted using a voltage of ~6.5 V/cm for 20 or 30 min. Fluorescent gel images of QDs and BSA were collected on a Bio-Rad Molecular Imager ChemiDoc XRS+ system (Bio-Rad Laboratories, Inc., Hercules, CA) using trans UV excitation. AuNP images were taken with a white LED back light and camera.

Results and discussion—Suppression of gel formation with amine-appended TA derivatives—TA-based NP ligands appended with an amine group tend to irreversibly transform into a sticky gel (more rapidly when stored neat or in highly concentrated solutions) that is difficult to redissolve in common organic solvents. The formation of an insoluble gel makes the subsequent processing quite difficult or almost impossible. Discoloration of the original pale yellow color of TA along with gel formation strongly suggests the ring opening polymerization of the 1,2-dithiolane unit. Since amine-appended TA derivatives are useful not only as a precursor for further synthetic transformation, but also as reactive surface ligands of QDs and AuNPs for a variety of bioconjugation strategies such as EDC coupling, it is beneficial to keep the material handling process of amine-appended TA derivatives easy and trouble free. Suppressing the gel formation of amine-appended TA derivatives in a simple manner was explored. Here, trifluoroacetamide was applied as the amine protecting group to the amine-appended TA derivatives. The results show that the trifluoroacetamide protecting group suppresses gel formation during ligand storage and is easily removed via deprotection under mild conditions to prepare amine-functionalized NPs.

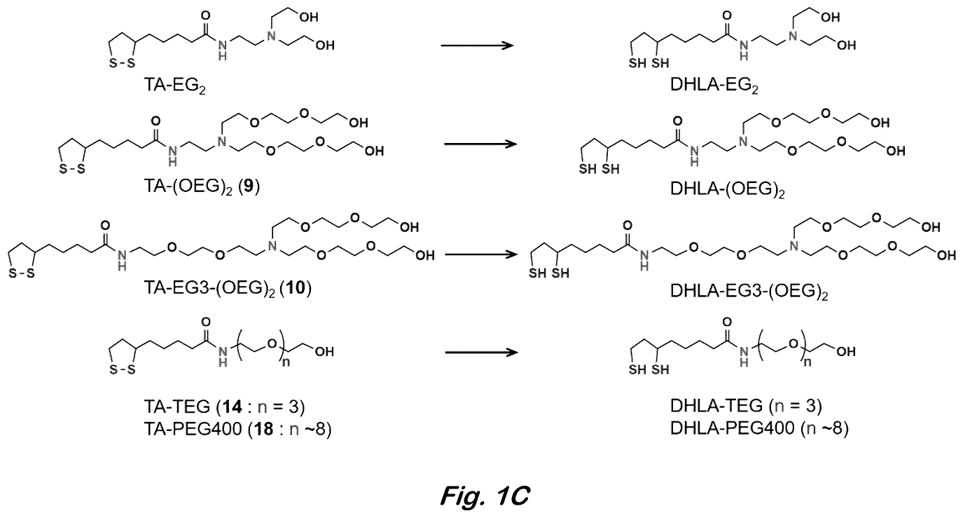

Among a variety of amine protecting groups, the protection with trifluoroacetamide formation can be simply done with ethyl trifluoroacetate at room temperature (FIG. 2A). Trifluoroacetamide group is relatively labile and can be easily cleaved under mild basic conditions. The simple procedures and mild conditions of both protection and deprotection of the amine group with trifluoroacetamide allow handling of amine-appended TA derivatives with minimum effort. This protecting group also helps mitigate irreversible gel formation of amine-appended TA derivatives. This allows handling them without solubility issues and weighing the amount accurately. The quantitative material handling is important when you set up subsequent reactions or systematically control different ligand ratios on NP surfaces, both of which may require precisely controlled stoichiometries.

For example, N-(2-aminoethyl)-1,2-dithiolane-3-pentanamide (TA-NH₂) is transformed to a sticky gel once the product is dried after purification, which makes it difficult to further process the material and weigh the amount in a reliable manner for any quantitative experiments. On the other hand, TA-NH(COCF₃) is obtained as pale yellow powder. The powder product may be kept at room temperature over years without any visible change. TA-PEG-NH(COCF₃) derivatives were obtained as oil with no apparent sign of gel formation. Since the trifluoroacetamide group can be removed under mild basic conditions, the TA-NH(COCF₃) and TA-PEG-NH(COCF₃) derivatives can be directly transformed to the deprotected (free amine-appended) DHLA derivatives during the ligand exchange procedures (FIG. 1A). The transformation from TA-NH(COCF₃) to DHLA-NH₂ was independently confirmed by ¹H-NMR and mass spectral characterization.

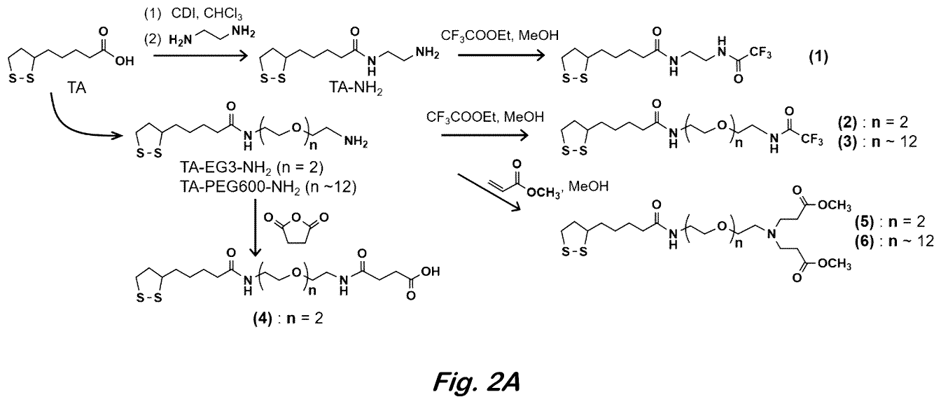

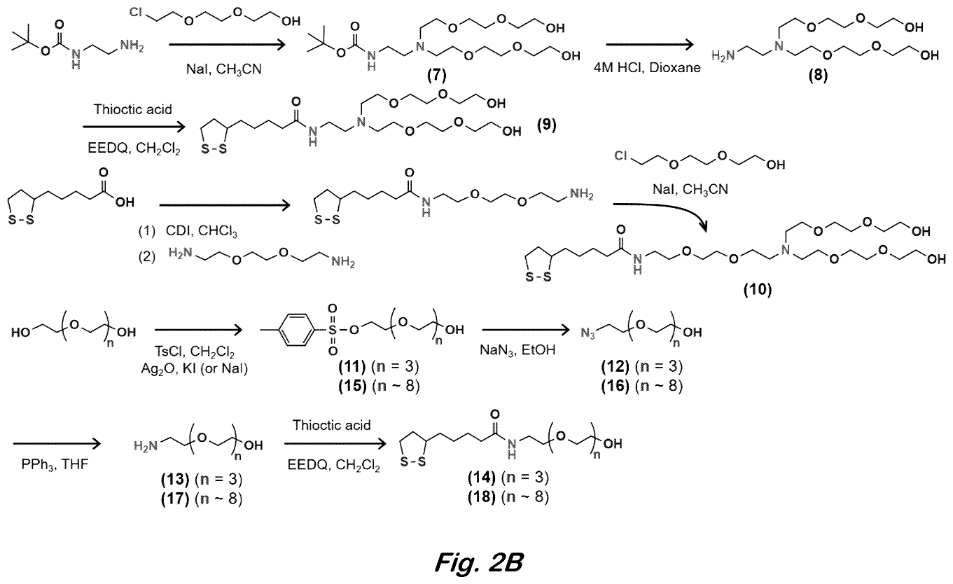

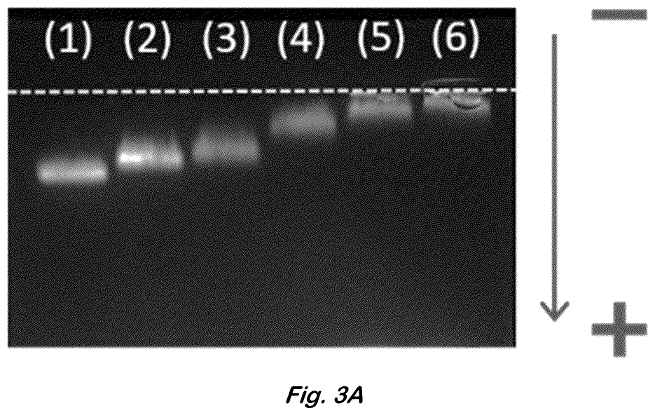

In order to demonstrate in situ deprotection of the trifluoroacetamide group on the amine-appended TA derivatives and the subsequent preparation of amine-modified QDs, two sets of QD samples were prepared. First, 520 nm emitting CdSe/CdS/ZnS QDs coated with different ratios of DHLA-EG3-COOH and DHLA-EG3-NH₂ were prepared, and gel electrophoresis was performed to characterize the QD surface properties (FIG. 3A). Considering the gel buffer used is 1×TBE (pH~8.3), the deprotonated carboxyl groups are expected to be a major driving force for the gel mobility toward the anode. The gel mobilities clearly reflect the ratio of terminal carboxyl and amino groups of QD surface ligands: the QDs with higher carboxyl group ratio showed higher gel mobility toward the anode. This suggests that the starting material TA-EG3-NH(COCF₃) was transformed to DHLA-EG3-NH₂ during the ligand exchange procedure and modified the QD surface with ratiometric control. A higher ratio of DHLA-EG3-NH₂ and possibly the protonated forms are considered to contribute to the lower gel mobility of QDs toward the anode. In a second demonstration, 470 nm emitting ZnSe/Cd_{0.35}Zn_{0.65}S/ZnS QDs coated with (i) DHLA-PEG750-OMe:DHLA-PEG600-NH₂ (95:5) and (ii) DHLA-PEG750-OMe were prepared, and a coupling reaction with fluorescein isothiocyanate (FITC) was performed. The isothiocyanate group of FITC is reactive with amines and expected to be conjugated to DHLA-PEG600-NH₂ ligands bound on QD surfaces. Thus, QDs with amine-appended ligands will allow further modification of the QD surface with FITC, while the QDs coated only with DHLA-PEG750-OMe should show no reactivity and play a role as a control. During the FITC coupling, the reaction solution was clear and showed no sign of QD aggregation or precipitation. After the aqueous coupling reactions, the purification procedures were performed with a PD-10 desalting column (GE Healthcare) and Amicon Ultra centrifugal filters (MWCO 50K, Millipore) to remove the unreacted excess FITC. A new band appeared around 500 nm in the absorption spectrum of the amine-modified QDs after FITC coupling (FIG. 3B), which is ascribed as the absorption band of FITC, indicating the successful coupling between the amine terminal group and FITC. In other words, the amine terminal groups were exposed by deprotection of trifluoroacetamide group during the ligand exchange step and became available for conjugation as designed. The average number of FITC on the QD surface was estimated to be 4.9 based on the absorption spectral fitting. The control QD sample still showed trace amounts of FITC bound on the QD surface (FITC/QD~0.28), suggesting minor nonspecific adsorption of FITC in the present experimental condition. Since the photoluminescence (PL) of 470 nm emitting QDs and the absorption of FITC has sizable spectral overlap, the QD-FITC conjugates are expected to promote energy transfer from the QD to FITC. The PL spectrum of the QD-FITC conjugates exhibited significant decrease of the QD PL compared to that of the QD alone (FIG. 3C). This is concomitant with the enhancement of FITC fluorescence, indicating efficient energy transfer from QD to FITC via conjugate formation as expected. The energy transfer efficiency was estimated to be 0.84, which was calculated from the relative fluorescence intensity of the QD donor alone and the donor in the presence of acceptors using equation (1).

$$E = 1 - \frac{{F}_{DA}}{{F}_{D}} \qquad ( 1 )$$

Given the fluorescence quantum yield of the QD donor alone (0.33) and the spectral overlap integral (2.06×10⁻¹³ M⁻¹ cm³) calculated from equation (2), the Forster distance R₀ was estimated to be 4.82 nm from equation (3).

$$I = \frac{{\int }_{0}^{∞} {F}_{D} ( λ ) {ε}_{A} ( λ ) {λ}^{4} d λ}{{\int }_{0}^{∞} {F}_{D} ( λ ) d λ} \qquad ( 2 )$$

$${R}_{0}^{6} = \frac{9000 ( ln 10 ) {κ}^{2} {Φ}_{D}}{128 {π}^{5} {n}_{r}^{4} {N}_{A}} I \qquad ( 3 )$$

$$E = \frac{{nR}_{0}^{6}}{{nR}_{0}^{6} + {r}^{6}} \qquad ( 4 )$$

From equation (4), the calculated distance from the QD donor to FITC was 4.76 nm. Since the radius of the 470 nm emitting QD obtained from the TEM characterization is 3.5 nm, the FITC dyes are located at 1.26 nm from the QD surface. On the other hand, the hydrodynamic radius of the 470 nm emitting QDs coated with DHLA-PEG750-OMe was estimated to be 5.55 nm from the dynamic light scattering measurement. The combined data suggest that the FITC dyes are located within the PEG surface layer on QD surface, which is reasonable given the shorter chain length of DHLA-PEG600-NH₂.

OEG PEG and zwitterion hybrid ligands to enhance biocompatibility—The compact ligand CL4 (FIG. 1B) was originally designed to hold its zwitterionic character across a certain pH range and has been utilized to maintain the colloidal stability of QDs and AuNPs in biological media. The wide range of utility of this compact ligand has been successfully demonstrated including biological sensing, cellular imaging, and catalytic activity studies. In the synthetic scheme of CL4, the first coupling of TA with ethylenediamine can be simply replaced with diamino derivatives of OEG or PEG with different chain length to create OEG or PEG/zwitterion hybrid functional groups in a single ligand structure (DHLA-EG3-CL4 and DHLA-PEG600-CL4 in FIG. 1B, synthesis of their precursors 5 and 6 in FIG. 2A). This hybrid design can be beneficial since each functional group has been widely proven to enhance the colloidal stability and biocompatibility of NPs. The entire synthetic scheme requires no extra effort compared with that of the original CL4 ligand.

1,2-Bis(2-aminoethoxy)ethane is commercially available at a reasonable cost. The precursors of DHLA-EG3-CL4 and DHLA-PEG600-CL4, 5 and 6, can be synthesized just like the precursor of CL4, TA-CL4(OMe)₂ (FIG. 2A). Synthesis of all the CL4 derivatives starts from the modification of the carboxyl group of TA with a diamino OEG or PEG linker using 1,1′-carbonyldiimidazole (CDI) as a coupling reagent. Subsequent aza-Michael addition reactions of TA-EG3-NH₂ and TA-PEG600-NH₂ with methyl acrylate formed 5 and 6, respectively. The last two steps of the ligand synthetic procedures are (i) deprotection of the methyl ester group to yield the carboxyl group and (ii) the ring-opening reduction from a disulfide group to a dithiol group, which were performed in situ during the subsequent ligand exchange.

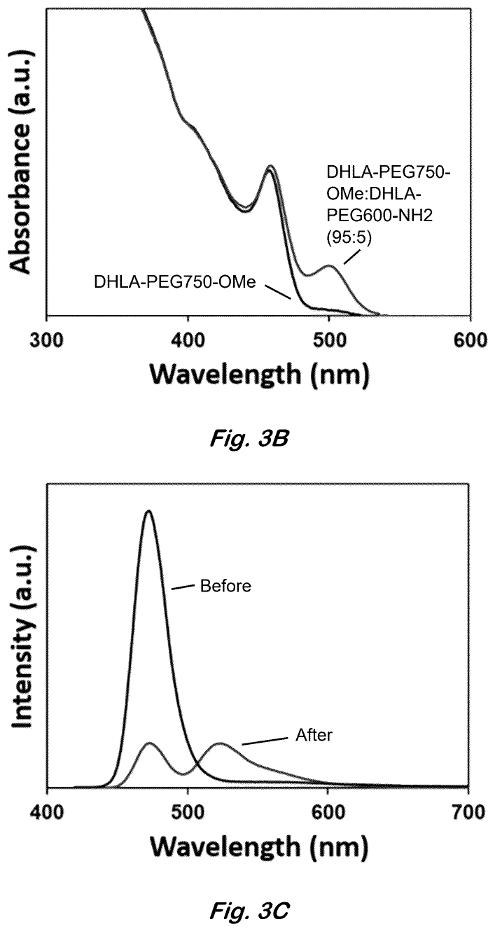

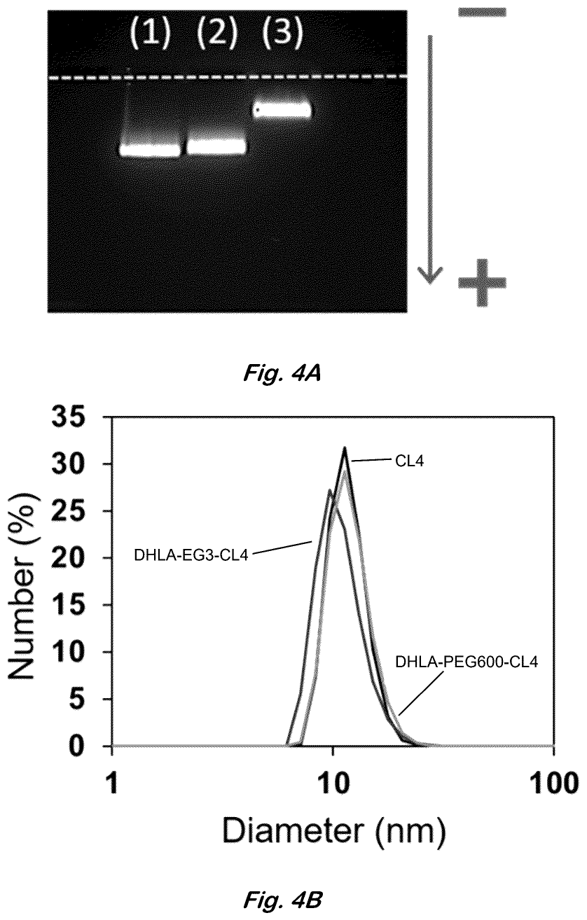

The QDs coated with CL4, DHLA-EG3-CL4 and DHLA-PEG600-CL4 were characterized by agarose gel electrophoresis (FIG. 4A). All the QDs moved toward the anode. Since the CL4 functional group (N,N-bis(2-carboxyethyl)amine group) has two carboxyl groups, net negative charges on QD surfaces are expected in 1×TBE buffer (pH 8.3). The gel mobility of DHLA-EG3-CL4-coated QDs was only slightly lower than that of CL4-coated QDs, and that of DHLA-PEG600-CL4-coated QDs was much lower than the other two. Given the fact that all the ligands terminate in CL4 functional group (two carboxyl groups and a tertiary amine group), the difference in gel mobility most likely originates from the mass difference of linkers connecting the TA unit and the bis(2-carboxyethyl)amine group.

The hydrodynamic diameters for the QD with CL4 derivatives were examined with dynamic light scattering analysis. The hydrodynamic diameters of the 515 nm emitting ZnSe/Cd_{0.4}Zn_{0.6}S/ZnS QDs coated with CL4, DHLA-EG3-CL4 and DHLA-PEG600-CL4 were 11.7±2.3, 10.9±2.8 and 12.0±2.7 nm, respectively (FIG. 4B). The inorganic diameter of the 515 nm emitting core-shell QD sample used in this analysis was measured by TEM to be 8.4±0.84 nm. The differences in sizes between hydrodynamic diameter and the inorganic core-shell size arise from hydrodynamic interactions of the ligand layer on the QD surface. The contribution of the surface ligands to the overall hydrodynamic radius is in the ~1.25-1.8 nm range, which is not too far from that estimated from just the simple fully extended geometric length of CL4 (~1.8 nm) but much smaller than those of DHLA-EG3-CL4 and DHLA-PEG600-CL4. It has been proposed that PEG chains can take on a mushroom-like conformation on NP surfaces depending on the ligand grafting density. The hydrodynamic size of DHLA-PEG600-CL4-coated QDs was not too different from those of CL4-QDs and DHLA-EG3-CL4-QDs, suggesting that DHLA-PEG600-CL4 units hold a mushroom-like conformation on QD surfaces, which minimize the hydrodynamic size in the present condition. The possible mushroom-like conformation of the PEG surface ligands implies a lower density of the surface ligands and could be partly due to the large surface curvature of QDs.

For biological applications of QDs, they need to be colloidally stable and capable of minimizing nonspecific adsorption of proteins in biological media. In order to further explore the utility of the OEG/PEG-CL4 hybrid ligands, the anti-fouling properties of QDs coated with the CL4 series were studied. Bovine serum albumin (BSA) was the protein of interest since BSA has been commonly utilized to assess the anti-fouling properties of a variety of water-soluble NPs. Minimization of nonspecific interaction of NPs with common proteins is considered to be ideal for NP target delivery and NP interaction with target receptors in cellular levels.

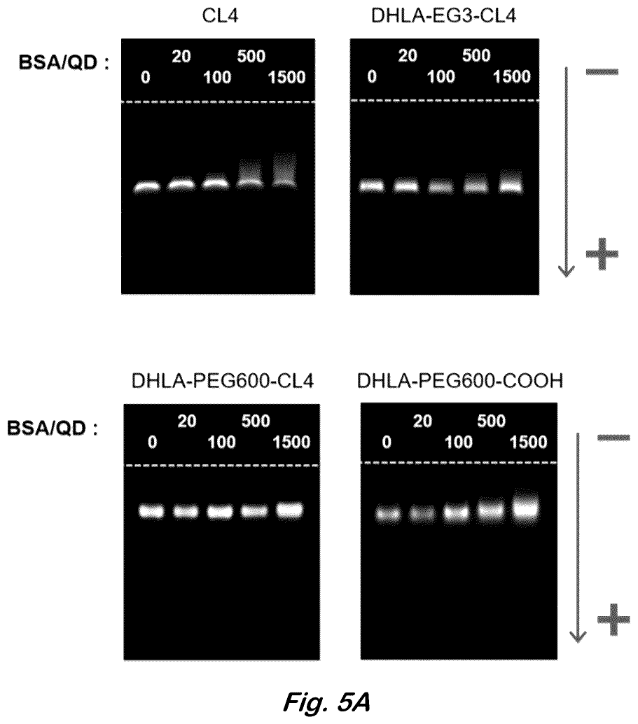

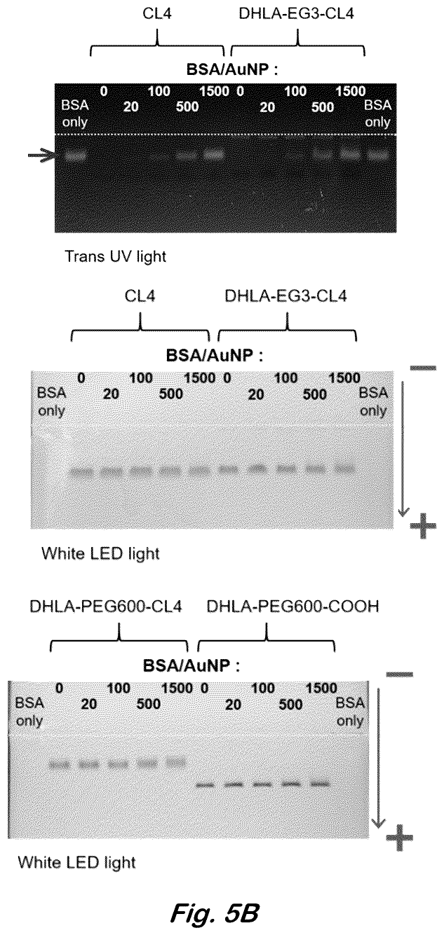

In these agarose gel electrophoresis assays, nonspecific adsorption of BSA on QD surface increases the overall size and mass of NPs, usually leading to the retardation of QD gel mobility. Each QD sample was incubated with BSA in 1×PBS buffer at a [BSA]/[QD] molar ratio ranging from 0 to 1,500 at room temperature for 30 min, followed by the gel electrophoresis assay (FIG. 5A). CL4-QDs showed a similar gel mobility up to [BSA]/[QD]=100. However, smearing of the QD bands was observed after [BSA]/[QD]=500, suggesting the nonspecific binding of BSA on the QD surfaces at higher BSA concentrations. DHLA-EG3-CL4-QDs also showed band smearing after [BSA]/[QD]=500, but the degree of smearing was significantly suppressed relative to CL4-QDs. In the case of DHLA-PEG600-CL4-QDs, there is no apparent change of gel mobility, and no smearing was observed up to [BSA]/[QD]=1,500. The result here clearly indicates that the insertion of OEG/PEG moiety helps suppress the nonspecific binding of BSAs on QD surfaces. On the other hand, DHLA-PEG600-COOH-QDs showed a slight broadening of gel bands at higher ratios of [BSA]/[QD]. The negatively charged carboxyl groups may still contribute to the nonspecific adsorption of BSA on QD surfaces. Clearly the combination of zwitterionic bis(2-carboxyethyl)amino terminal and OEG/PEG groups helps improve the anti-fouling properties in a synergistic manner.

The anti-fouling properties of AuNPs coated with the same ligand series were explored using the same gel electrophoresis assay format (FIG. 5B). The AuNPs were synthesized by the conventional seed growth method based on citrate reduction and stabilization, and the average size measured by TEM was ~7.8±1.5 nm. The citrate ligands on AuNPs were further replaced by each TA-based ligands using a ligand exchange technique following the deprotection of terminal methyl ester groups. It should be noted that the TA-based ligands were mixed with citrate-coated AuNPs without the ring opening step of the dithiolane group due to the much stronger intrinsic Au—S interaction. Each of the AuNPs coated with CL4-based ligands and DHLA-PEG600-COOH showed the same gel mobilities independent of [BSA]/[QD] ratio and no apparent sign of band retardation, indicating no BSA binding on the AuNP surface. The trend of gel mobility observed in AuNPs contrasts with that of QDs performed in the same conditions: the QDs coated with CL4 and DHLA-PEG600-COOH showed a degree of nonspecific BSA binding. A few possible reasons may be surmised that are related to the differences in surface chemistry between QDs and AuNPs and also their surface-ligand interactions. It is known that Au—S binding is stronger than Zn—S binding, and therefore, AuNPs can create a more stable surface coating with thiol-based ligands. The strong thiol affinity to gold atoms along with weak coordination of citrate ligands on AuNPs makes the ligand exchange highly efficient under mild conditions. On the other hand, QD synthesis often uses strong binding ligands for better size control. This possibly makes the subsequent ligand exchange of as-prepared QDs with thiol ligands less efficient and often requires harsher conditions. In addition, the ZnS outermost shell of QDs is a binary compound, and thiol ligands are expected to bind to Zn cations. While the synthesis was designed to create Zn-rich surfaces during the ZnS overcoating process, this does not guarantee a perfect Zn cation coverage on the QD surface and could result in a lower ligand surface coverage density of QDs versus that of AuNPs. Similar differences between QDs and AuNPs have been reported. The slight difference of anti-fouling properties between QDs and AuNPs is a reminder that judicious selection of surface ligands is more critical for QDs to maximize the anti-fouling properties.

Hydroxy-terminated OEG PEG ligands with a simple synthetic scheme—OEG or PEG derivatives have been utilized as hydrophilic surface coating materials for a wide variety of NPs for biological applications. In order to overcoat NPs, they are usually appended with surface binding groups (e.g., thiol) or polymeric scaffolds. There are not many asymmetrically substituted OEG or PEG derivatives commercially available with reasonable cost. This is probably because most of the asymmetric OEG or PEG derivatives are transformed from regular symmetric OEGs or PEGs, and the transformation mostly requires tedious column purification with multistep synthesis. As a matter of fact, OEG/PEG-based surface coating ligands with intact terminal hydroxy groups are not very common.

A hydroxy group is one of the common hydrophilic functional groups which can help maintain the colloidal stability of QDs and AuNPs in aqueous media. Hydroxy groups coated on NPs can create hydrogen bond networks with the surrounding water molecules and provide a hydrating layer. The hydrating layer is expected to stabilize the colloids against aggregation and hinder protein adsorption. A variety of hydroxy-terminated ligands including mono- or oligosaccharide and PEG based ligands have been reported to prepare water-soluble QDs and AuNPs. However, there are fewer reports of hydroxy terminated ligands for stabilizing QDs and AuNPs compared to those that are modified with terminal capped PEG or zwitterionic functional groups. This is likely due to the potential reactivity of hydroxyl groups during multistep ligand syntheses, where unprotected hydroxy groups would limit ligand design. Typical symmetric OEGs and PEGs have two hydroxy groups at both ends. In order to keep the synthetic design of hydroxy-terminated OEG/PEG ligands simple, only one of the two terminal hydroxy groups is modified with the surface binding group, and the other hydroxy group is left intact. However, this apparently simple synthetic scheme usually comes with tedious column purification procedures. While the use of protecting groups for hydroxy groups can be integrated in the synthetic scheme, that usually requires extra two steps (protection and deprotection), which is not ideal to simplify the ligand synthetic scheme and could lower the final product yield.

There are previously designed and synthesized PEG based ligands appended with TA at one end and a variety of functional groups at another end (Susumu et al., J. Am. Chem. Soc. 2007, 129, 13987-13996; Mei et al., J. Mater. Chem. 2008, 18, 4949-4958). Those ligands have been successfully utilized for a series of biological sensing, imaging and enzymatic activity studies. In the present study, one terminal hydroxy group of OEG or PEG was left as an additional benefit for hydrophilicity and colloidal stability of QDs and AuNPs in aqueous media. Symmetric OEG or PEG has been directly coupled with TA via an ester bond to synthesize TA-PEG-OH ligands that have a terminal hydroxy group. However, the labile nature of ester linkages is not an ideal choice for a wide range of biological applications. On the other hand, an amide linkage is robust and tolerates even harsh synthetic conditions and biological environments.

It was found that 2-[2-(2-chloroethoxy)ethoxy]ethanol, an asymmetric OEG derivative, is widely available at reasonable cost on the market. Since the terminal chloride can be transformed into iodide and subsequently substituted with an amine group, compound 9 (TA-(OEG)₂) was designed and synthesized as a surface ligand for QDs and AuNPs (FIG. 2B). This ligand consists of two branched hydroxy-terminated OEG groups, which are expected to provide reasonable colloidal stability in aqueous media despite the compact ligand size. While its chemical structure appears complicated, the synthesis of 9 takes only 3 steps, and the first 2 steps can be done in high yield without relying on column chromatography. The amide coupling reaction between TA and compound 8 was performed with N-ethoxycarbonyl-2-ethoxy-1,2-dihydroquinoline (EEDQ). EEDQ is efficient for amide bond formation in the presence of unprotected hydroxy groups, and therefore, is an ideal choice of coupling reagent without compromising the simple synthetic scheme. Compound 14 (TA-TEG) was synthesized as a control since it contains a single tetra(ethylene glycol) (TEG) group (not branched) and the molecular length of 14 is similar to 9. The synthesis of 14 takes 4 steps in total including the cumbersome purification step of the asymmetric OEG precursor 11. Ligand 10 is structurally similar to 9, however, 1,2-bis(2-aminoethoxy)ethane was used as a central module rather than ethylenediamine for 9. The synthetic scheme used to make compound 9 was simplified to prepare compound 10 in only two consecutive steps. Ligand 10 has 9 ethylene oxide units in total, which are similar to PEG400. For comparison, TA-PEG400 18 was synthesized as well following the synthetic procedure of 14. All these ligands were transformed into the DHLA derivatives in situ during the ligand exchange process.

The hydrodynamic diameters of the 520 nm emitting CdSe/Cd_{0.75}Zn_{0.25}S/ZnS QDs coated with DHLA-TEG, DHLA-(OEG)₂, DHLA-PEG400, DHLA-EG3-(OEG)₂ were 7.3±1.6, 6.0±1.4, 6.7±1.6 and 7.0±1.7 nm, respectively. The diameter of the 520 nm emitting core-shell QD (CdSe/Cd_{0.75}Zn_{0.25}S/ZnS) sample used in this analysis was measured by TEM to be 4.4±0.5 nm. The hydrodynamic diameters of the QDs coated with these hydroxy-terminated ligands are overall in a similar range. This trend is rather similar to the results observed in the CL4 series discussed above, suggesting that each ligand keeps their conformation compact on QD surface to minimize the hydrodynamic size.

The long-term colloidal stability of QDs coated with the series of hydroxy-terminated ligands DHLA-TEG, DHLA-(OEG)₂, DHLA-PEG400, DHLA-EG3-(OEG)₂ was examined across a wide pH range. The luminescence images of 520 nm emitting QDs coated with those hydroxy-terminated ligands dispersed in buffer solutions from pH 2 to 13 were periodically taken from <20 min to 2 months after the sample preparation. The samples were stored in a refrigerator during the interval. Gradual fluorescence quenching of the QDs at strongly acidic conditions (pH<3~4) was observed, similar to the trends observed for QDs coated with the other DHLA-based ligands at the same pH. As discussed before, this strongly acidic environment most likely causes QD surface etching or dissociation of the ligand-QD bonds via protonation of thiolate groups. The remaining QD dispersions were colloidally stable in weakly acidic to strongly basic pH conditions (pH 4-5 to 12-13) without apparent fluorescence quenching for at least a 2 month period. The ligands with longer ethylene oxide repeat units appear to perform better and help maintain the QD colloidal stability longer near the more extreme pH values. The number of hydrophilic ethylene oxide repeating units in DHLA-(OEG)₂ and DHLA-EG3-(OEG)₂ is 6 and 9, respectively. The pH stability of the QDs coated with DHLA-(OEG)₂ and DHLA-EG3-(OEG)₂ is still comparable to that of QDs coated with DHLA-PEG750-OMe ligands (~16 ethylene oxide units) where the neutral ethylene oxide repeat units solely mediate aqueous solubility. The comparison here suggests that both the ethylene oxide repeating units of OEG/PEG and the terminal hydroxy groups help enhance the pH stability of the QDs coated with these ligands. The colloidal stability of the same QD series was examined under high salt concentration as well. Each QD was dispersed in 3 M NaCl solutions (pH ~6), stored in a refrigerator, and the fluorescence images were recorded periodically. As expected from previous studies, all the QDs appeared stable and well dispersed for at least 2 months without any apparent sign of aggregation or quenching. The physiological NaCl concentration is generally reported to be ~150 mM, and biological environments are also known to be rich in a variety of other strong electrolytes. Given the colloidal stability in much higher NaCl concentration, the QDs coated with these hydroxyl-terminated ligands are expected to be tolerant of similar ion-rich biological environments for a long period of time.

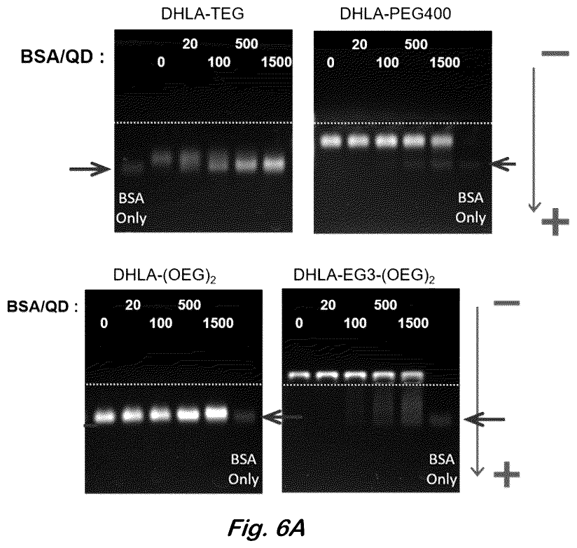

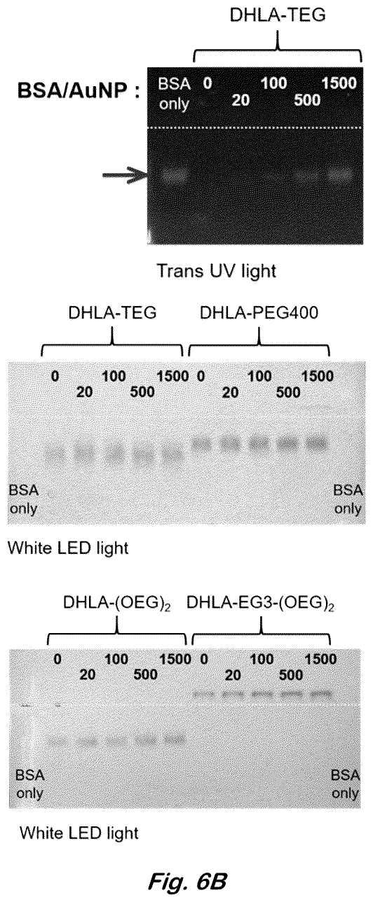

In order to assess the anti-fouling properties, gel electrophoresis assay of QDs coated with each hydroxy terminated ligand DHLA-TEG, DHLA-(OEG)₂, DHLA-PEG400, DHLA-EG3-(OEG)₂ was performed in the presence of incremental ratios of BSA under the same conditions as described above (FIG. 6A). Free DHLA-TEG coated QDs and BSA run closely, with BSA running slightly closer to the anode. As the [BSA]/[QD] ratio increases, the free QD bands gradually fade away concomitant with the increase of a band closely overlapped with BSA. This suggests nonspecific binding of BSA as the [BSA]/[QD] ratio increases. The gel mobility of the free QDs is relatively low due to the intrinsically neutral structure, and the BSAs bound on the QD surface seem to drag the QD and slightly enhance the gel mobility. On the other hand, the gel mobility of DHLA-PEG400 coated QDs shows no apparent change as the [BSA]/[QD] ratio increases, indicating excellent anti-fouling behavior. Since the gel mobility of DHLA-PEG400 coated QDs is lower than that of DHLA-TEG coated QDs, the clear band separation of BSA can be observed. The comparison of these two ligands indicates that PEG needs to have a certain chain length to fully ensure the anti-fouling character of QDs used. DHLA-(OEG)₂ coated QDs showed gel mobility similar to that of BSA. As the [BSA]/[QD] ratio increases, the bands still showed the same mobility and no clear sign of new band formation. Interestingly, DHLA-EG3-(OEG)₂ coated QDs moved slightly toward the cathode, suggesting the QD surface was positively charged. While they seem to have overall good anti-fouling character, trace amounts of streaky bands were observed as the [BSA]/[QD] ratios increased.

The gel electrophoresis assay with the same format was also performed with the AuNPs (~7.8 nm in diameter) coated with the same ligands (FIG. 6B). Each ligand series with different [BSA]/[AuNP] ratios still showed the same gel mobilities without any apparent sign of nonspecific adsorption of BSA up to [BSA]/[AuNP]=1,500. The present gel electrophoretic assay shows that the relatively short OEG/PEG ligands are still effective to suppress nonspecific binding of BSA on AuNPs. As discussed above, further improved anti-fouling behavior of AuNPs compared with QDs coated with the same ligands would be due to (i) stronger Au—S binding and (ii) higher ligand density on AuNP surface. Overall, the present study suggests that not only the OEG/PEG units but also the terminal hydroxy groups contribute to the suppression of nonspecific binding of BSA.

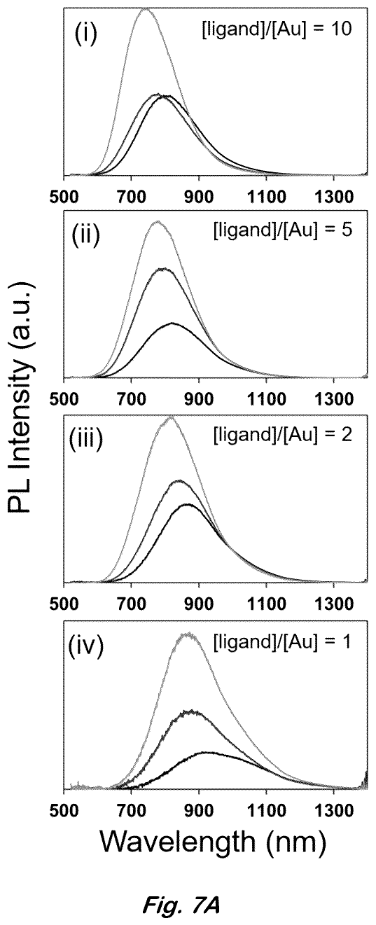

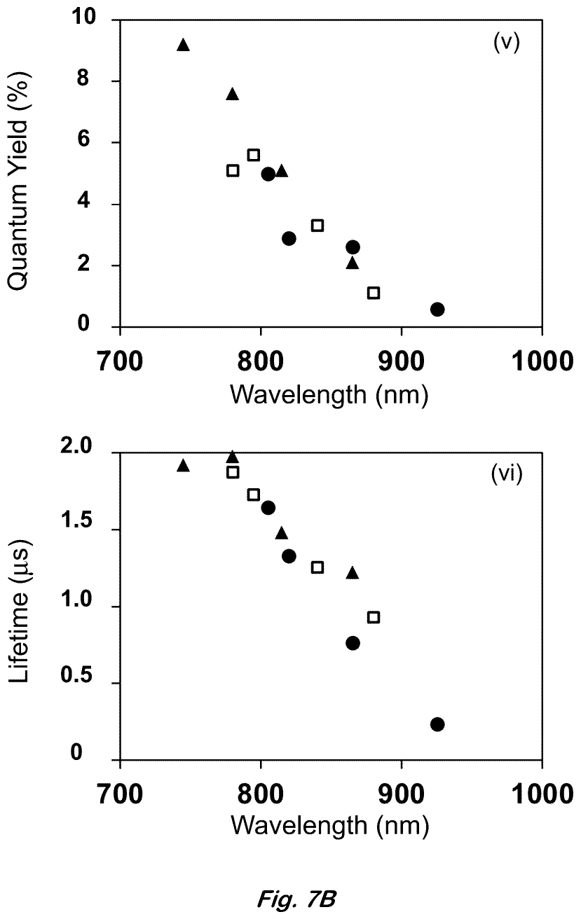

Direct synthesis of luminescent gold nanoclusters with hydroxy terminated ligands—Near infrared (IR) luminescent gold nanoclusters (AuNCs) were previously synthesized in a one-step aqueous phase condition using a series of PEGylated TA ligands with a methoxy, amine, carboxy, or azide terminal group. AuNCs have been particularly attractive as biological probes due to their small core size (<2 nm), near IR luminescence, and the intrinsic non-toxicity of gold. In order to demonstrate further utility of the new ligands, a direct aqueous phase synthesis of near IR luminescent AuNCs was performed using the hydroxy-terminated ligands: TA-EG₂, TA-(OEG)₂ and TA-EG3-(OEG)₂. The ligand TA-EG₂ was previously developed as a hydrophilic compact ligand (FIG. 1C). The chemical structure of TA-EG₂ is similar to TA-(OEG)₂ and TA-EG3-(OEG)₂: they all have two hydroxy terminal groups branched from a tertiary amine. In addition, the hydrophilicity and biocompatibility of TA-EG₂ is as promising as the other hydroxyl-terminated ligands. The ligand TA-EG₂ is utilized to help discuss the relationship between the ligand structure and AuNC core growth. The AuNCs were synthesized at room temperature using NaBH₄ as a reducing agent. In order to monitor the influence of [ligand]/[Au] molar ratio on the core growth, the amount of each ligand was systematically changed. The amount of all the other reagents was kept constant. It has been demonstrated that the size (or PL peak) of AuNCs can be tuned by adjusting the [ligand]/[Au] molar ratio, and [ligand]/[Au] ratios=1, 2, 5 and 10 were explored in the present study. The absorption spectra of AuNCs with those hydroxy-terminated ligands showed features similar to those of AuNCs prepared with TA-PEG ligands. The absorbance decreases continuously from UV to near IR with some shoulder peaks present. As the [ligand]/[Au] ratio decreases, the absorption onsets were further red-shifted, indicating a decrease in the band gap. PL spectra of the AuNCs were also measured by excitation at 500 nm (FIG. 7A). Interestingly, the PL peak positions were dependent on both the [ligand]/[Au] molar ratio and the ligand structure. For each ligand, a blue shift of the PL peak position occurs as the [ligand]/[Au] molar ratio increases, as has been observed in other TA-based ligands. The PL peak positions were blue-shifted from the [ligand]/[Au] ratio 1 to 10 as follows: TA-EG₂, ~925 nm to ~805 nm; TA-(OEG)₂, ~880 nm to ~780 nm; TA-EG3-(OEG)₂, ~865 nm to ~745 nm. From this data it can be seen that at the same [ligand]/[Au] molar ratio, the AuNC PL wavelength is more blue-shifted as the size of the ligand used in the synthesis is larger. The present results evince that the core growth of AuNCs were controlled by the [ligand]/[Au] molar ratio and also by the size of the TA-based ligands. The TA-based ligands are initially coordinated to gold cations before the cluster growth is initiated by the addition of reducing agent. It is speculated that the larger ligands possibly suppress the efficient collision of gold atoms for cluster growth and control the reaction rate and eventually make the final cluster size smaller. The AuNC size was measured by TEM to assess this hypothesis. The average inorganic core diameters of AuNCs prepared by [ligand]/[Au] ratio=2 with TA-EG₂, TA-(OEG)₂ and TA-EG3-(OEG)₂ were 1.82±0.40, 1.78±0.26 and 1.45±0.21 nm, respectively. Although the size difference is trivial, the present data set simply shows that the larger AuNC cores have more red-shifted PL peaks, suggesting that quantum size effect plays a role in the PL peak positions of AuNCs during the core growth. PL quantum yields of the AuNCs are in the range similar to previous reports for a series of TA-PEG ligands. The red shifts of PL peaks are followed by the decrease in PL quantum yields (FIG. 7B). This trend seems to align with the energy gap law. PL lifetimes of the AuNCs were also measured using time-correlated single photon counting (TCSPC) technique. The PL decay curves were fitted as tri-exponential decay, and the intensity-weighted average lifetimes were used for comparison. The range of lifetimes (1~2 μs or less) is also similar to those measured in the previous study with TA-PEG ligands and other AuNCs coated with thiol based ligands. For the same [ligand]/[Au] ratio, the average lifetimes consistently increased in the following order: TA-EG₂<TA-(OEG)₂<TA-EG3-(OEG)₂ (FIG. 7B). The higher QYs are correlated with the longer average lifetimes. This simply indicates that the nonradiative decay rate constants decreased as the PL peaks are blue-shifted, which agrees with the energy gap law. While the PL mechanism of AuNCs is still under debate, the present data set suggests that the quantum size effect plays some role in the PL mechanism of AuNCs prepared in this study. The present synthetic conditions allowed tuning the PL peaks in the near IR region from 745 to 925 nm just by adjusting the [ligand]/[Au] ratios and modification of the ligand structures. Given the promising biological utility of QDs and AuNPs coated with the hydroxy-terminated ligands studied above, the successful preparation and PL tuning of near IR luminescent AuNCs would make the hydroxyl-terminated compact ligands worth exploring for more sophisticated biological applications in the area of in vivo sensing and imaging. Near IR luminescent AuNCs with compact hydrophilic ligands are particularly promising for in vivo study due to the low toxicity, compact overall size and high penetration depth of near IR light in deep tissue.

Many modifications and variations are possible in light of the above teachings. It is therefore to be understood that the claimed subject matter may be practiced otherwise than as specifically described. Any reference to claim elements in the singular, e.g., using the articles “a”, “an”, “the”, or “said” is not construed as limiting the element to the singular.

What is claimed is:

1. A compound having the formula: 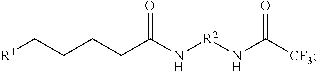
wherein R¹ is 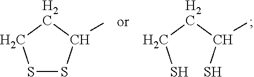
and
wherein R² is an organic group.

2. The compound of claim 1, wherein the compound is

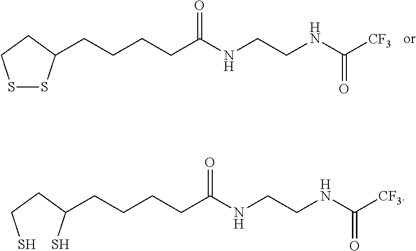 [SMILES: O=C(CCCCC1CCSS1)NCCNC(=O)C(F)(F)F ; O=C(CCCCC(S)CCS)NCCNC(=O)C(F)(F)F]

3. The compound of claim 1;
wherein the compound is 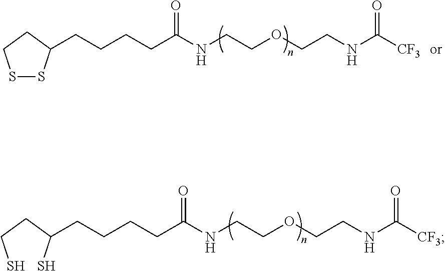
and
wherein n is a positive integer.

4. The compound of claim 3, wherein n is from 2 to 12.

5. A composition comprising:
a nanoparticle; and
the compound of claim 1;
wherein the sulfur atoms of the compound are bound to the nanoparticle.

6. The composition of claim 5, wherein the nanoparticle comprises a semiconducting material.

7. The composition of claim 5, wherein the nanoparticle comprises CdS, ZnS, Ag₂S, PbS, HgS, CdSe, ZnSe, Ag₂Se, PbSe, HgSe, CdTe, ZnTe, Ag₂Te, PbTe, HgTe, CuInS₂, CuInSe₂, InP, InAs, or a combination thereof.

8. The composition of claim 5, wherein the nanoparticle comprises gold, silver, copper, platinum, or palladium.

9. A method comprising:
providing an amine have the formula: 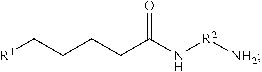
wherein R¹ is 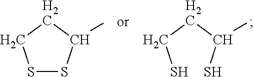
and
wherein R² is an organic group; and
reacting the amine with ethyl trifluoroacetate to form a compound having the formula:

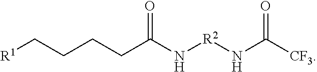

10. The method of claim 9, wherein R² is —CH₂—CH₂—.

11. The method of claim 9;
wherein R² is —(CH₂—CH₂—O)ₙ—CH₂—CH₂—; and
wherein n is a positive integer.

12. The method of claim 11, wherein n is from 2 to 12.

13. The method of claim 9, further comprising:
binding the sulfur atoms of the compound to a nanoparticle.

14. The method of claim 13, wherein the nanoparticle comprises a semiconducting material.

15. The method of claim 13, wherein the nanoparticle comprises CdS, ZnS, Ag₂S, PbS, HgS, CdSe, ZnSe, Ag₂Se, PbSe, HgSe, CdTe, ZnTe, Ag₂Te, PbTe, HgTe, CuInS₂, CuInSe₂, InP, InAs, or a combination thereof.

16. The method of claim 13, wherein the nanoparticle comprises gold, silver, copper, platinum or palladium.

17. A compound having the formula: 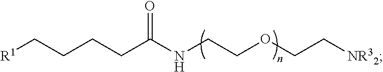
wherein n is a positive integer
wherein R¹ is 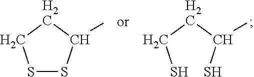
wherein each R³ is —H, —[(CH₂)₂—O]ₘ—(CH₂)₂—OH or —(CH₂)₂—CO—O—R⁴;
wherein at most one of the R³ groups is —H;
wherein m is a positive integer; and
wherein R⁴ is an alkyl group or H.

18. The compound of claim 17, wherein the compound is

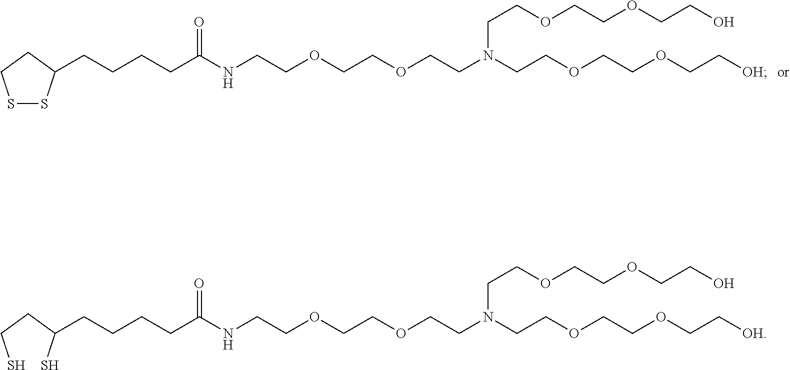 [SMILES: O=C(CCCCC1CCSS1)NCCOCCOCCN(CCOCCOCCO)CCOCCOCCO ; O=C(CCCCC(S)CCS)NCCOCCOCCN(CCOCCOCCO)CCOCCOCCO]

19. The compound of claim 17, wherein the compound is

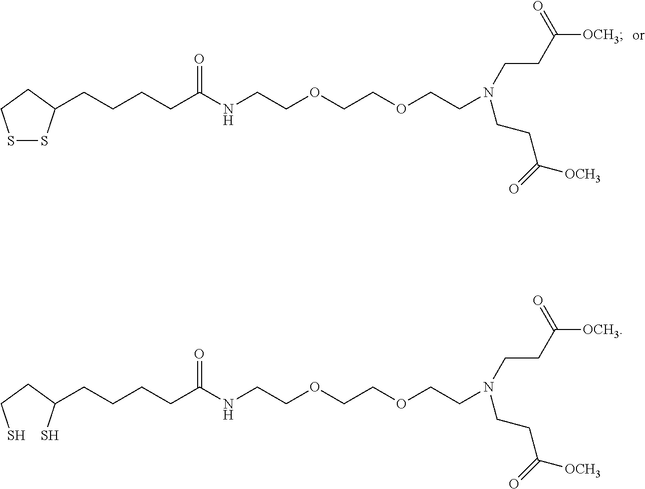 [SMILES: COC(=O)CCN(CCOCCOCCNC(=O)CCCCC1CCSS1)CCC(=O)OC ; COC(=O)CCN(CCOCCOCCNC(=O)CCCCC(S)CCS)CCC(=O)OC]

20. A composition comprising:
a nanoparticle; and
the compound of claim 17;
wherein the sulfur atoms of the compound are bound to the nanoparticle.

21. The composition of claim 20, wherein the nanoparticle comprises a semiconducting material.

22. The composition of claim 20, wherein the nanoparticle comprises CdS, ZnS, Ag₂S, PbS, HgS, CdSe, ZnSe, Ag₂Se, PbSe, HgSe, CdTe, ZnTe, Ag₂Te, PbTe, HgTe, CuInS₂, CuInSe₂, InP, InAs, or a combination thereof.

23. The composition of claim 20, wherein the nanoparticle comprises gold, silver, copper, platinum, or palladium.
